(19) United States
(12) Patent Application Publication
(10) Pub. No.: US 2024/0058390 A1
(43) Pub. Date: Feb. 22, 2024

(54) WNT+ ADIPOCYTES, EXOSOMES FROM WNT+ ADIPOCYTES, AND METHODS OF MAKING AND USING THEM

(71) Applicant: The Administrators of the Tulane Educational Fund, New Orleans, LA (US)

(72) Inventors: YiPing CHEN, Harahan, LA (US); Zhi LIU, Chengdu (CN); Tian CHEN, Chengdu (CN); Sicheng ZHANG, Chengdu (CN)

(73) Assignee: The Administrators of the Tulane Educational Fund, New Orleans, LA (US)

(21) Appl. No.: 18/266,986

(22) PCT Filed: Dec. 16, 2021
(86) PCT No.: PCT/US21/63907

§ 371 Date: Jun. 13, 2023

Related U.S. Application Data
(60) Provisional application No. 63/126,170, filed on Dec. 16, 2020.

(51) Int. Cl. A61K 35/35; A61K 9/127; A61K 38/28; A61P 3/10; C12N 5/077

(52) U.S. Cl. CPC A61K 35/35; A61K 9/127; A61K 38/28; A61P 3/10; C12N 5/0653; C12N 2506/1353; C12N 2510/00

(57) ABSTRACT

The invention relates to the discovery of a previously unknown population of adipocytes marked by active intracellular Wnt/β-catenin signaling. The present invention provides methods of making populations of adipocytes in which the proportion of these “Wnt⁺ adipocytes” is much higher than those in naturally occurring populations. Wnt⁺ adipocytes can be used to improve blood glucose control and exosomes secreted by Wnt⁺ adipocytes can be administered to reduce blood glucose levels in subjects in need thereof.

### CROSS-REFERENCE TO RELATED APPLICATIONS

This application claims the benefit of U.S. Provisional Patent Application Nos. 63/126,170, filed Dec. 16, 2020, the contents of which are incorporated herein by reference for all purposes.

### STATEMENT OF FEDERAL FUNDING

This invention was made with government support under RO1DK128907, awarded by the National Institute of Diabetes and Digestive and Kidney Diseases of the National Institutes of Health. The government has certain rights in the invention.

### BACKGROUND OF THE INVENTION

Adipose tissues, consisting of white adipose tissue (“WAT”) and brown adipose tissue (“BAT”), play a critical role in maintaining whole-body metabolic homeostasis, with WAT serving for energy storage and BAT for energy dissipation to produce heat. (See, e.g., Cannon and Nedergaard, Physiol Rev, 2004, 84:277-359, doi:10.1152/physrev.00015.2003; Rosen and Spiegelman, Cell, 2014, 156:20-44, doi:10.1016/j.cell.2013.12.012). In addition to white and brown fat cells that are differentiated from heterogenous stromal vascular fractions (SVFs) of distinct lineages (Hepler, et al., Genes Dev, 2017, 31:127-140, doi:10.1101/gad.293704.116; Rosenwald and Wolfrum, Adipocyte, 2014, 3:4-9, doi:10.4161/adip.26232; Schwalie, et al., Nature, 2018, 559:103-108, doi:10.1038/s41586-018-0226-8), a third type of inducible adipocyte exists known as beige adipocytes that are transiently generated in WAT depots in response to external stimulations such as environmental cold acclimation (Bostrom, et al., Nature, 2012, 481:463-468, doi:10.1038/nature10777; Ikeda, et al., Trends Endocrinol Metab. 2018, 29:191-200, doi:10.1016/j.tem.2018.01.001; Wu, J. et al.et al., Cell, 2012, 150:366-376, doi:10.1016/j.cell.2012.05.016. Like brown adipocytes, activated beige adipocytes through “beiging” or “browning” process of WAT express key thermogenic marker uncoupling protein 1 (UCP1) and exert adaptive thermogenic function (Ishibashi and Seal, Science, 2010, 328:1113-1114, doi:10.1126/science.1190816; Petrovic, et al., J Biol Chem. 2010, 285:7153-71649). Although the capacity for thermogenic fat cells (i.e., brown and beige adipocytes) to protect against diet-induced obesity and metabolic disorders has been recognized (see, e.g., Bartelt and Heeren, Nat Rev Endocrinol, 2014, 10:24-36, doi:10.1038/nrendo.2013.204; Crane, Nat Med, 2015, 21:166-172, doi:10.1038/nm.3766), a comprehensive understanding of developmental origins and regulatory mechanism of beiging is still lacking, partially due to the cell-type complexity in adipose tissues. It has been appreciated that beige adipocytes arise by both de novo adipogenesis from progenitor cells and direct conversion/transdifferentiation from white adipocytes (see, e.g., Barbatelli, et al., Am J Physiol Endocrinol Metab, 2010, 298:E1244-1253, doi:10.1152/ajpendo.00600.2009; Shao, et al., Diabetes, 2019, 68:1874-1885, doi:10.2337/db19-0308). While previous profiling studies have identified some specific markers (for example, CD137 and CD81) for progenitors of beige adipocytes (see, e.g., Wu, et al., supra; Oguri, et al., Cell, 2020, 182:563-577 e520, doi:10.1016/j.cell.2020.06.021), the cellular origin of beige adipocytes derived from direct conversion remained elusive.

The Wnt/β-catenin (canonical) signaling pathway plays a fundamental role in cell proliferation, differentiation, and tissue homeostasis. In the presence of Wnts, a “destruction complex” including glycogen synthase kinase 3 (GSK-3) is inhibited and cytoplasmic β-catenin translocates into the nucleus and interacts with TCF/LEF transcription factors to activate downstream target genes. In addition, β-catenin nuclear translocation can also be triggered by other intracellular factors such as AKT and Gαs, leading to the activation of Wnt/β-catenin signaling in a ligand- and receptor-independent manner Despite the consensus that Wnt/β-catenin signaling imposes negative effects on adipogenesis by inhibiting Pparγ, clues have pointed to potentially crucial roles of Wnt/β-catenin signaling in adipogenesis and adipose tissue function. For instance, β-catenin and Tcf712, two key effectors of canonical Wnt signaling pathway, are expressed by mature adipocytes. Genetic mutations in Ctnnb1 (encoding β-catenin) and Tcf712 lead to impaired adipocyte development and maturation (e.g., Chen, et al, 2020, Sci Adv 6:eaax9605, doi:10.1126/sciadv.aax9605; Chen, et al., Diabetes, 2018, 67:554-568, doi:10.2337/db17-0318). In particular, loss of Tcf712 in mature adipocytes gives rise to adipocyte hypertrophy, inflammation, as well as systemic glucose intolerance and insulin resistance, implying an important biological role for Wnt/β-catenin signaling in adipose function (Chen, et al., 2018, supra, Geoghegan et al., Mol Metab, 2019, 24:44-63, doi:10.1016/j.molmet.2019.03.003).

It would be desirable to have additional compositions that ameliorate some of the symptoms and effects of type 1 diabetes mellitus, type 2 diabetes mellitus, or both. It would further be desirable to have methods to produce those compositions. Surprisingly, the present invention fulfills these and other needs.

### BRIEF SUMMARY OF THE INVENTION

In a first group of embodiments, the invention provides populations of exosomes secreted by a population containing Wnt⁺ adiopocytes in percentages higher than those that exist in any naturally-occurring population of adipocytes. In some embodiments, at least 30% of the population of adipocytes are Wnt⁺ adiopocytes. In some embodiments, at least 50% of the adipocytes are Wnt⁺ adiopocytes. In some embodiments, at least 90% of the adipocytes are Wnt⁺ adiopocytes. In some embodiments, 95% or more of the adipocytes are Wnt⁺ adiopocytes. In some embodiments, the Wnt⁺ adiopocytes are white adipocytes. In some embodiments, the adipocytes are human adipocytes.

In another group of embodiments, the invention provides uses for populations of exosomes derived from a population of adipocytes of which at least 30% of adipocytes are Wnt⁺ adiopocytes, for use to improve insulin production, to reduce insulin resistance in a subject in need thereof, to treat hepatic steatosis, or to treat metabolic syndrome. In some embodiments, at least 50% of the adipocytes in said population are Wnt⁺ adiopocytes. In some embodiments, at least 90% of said adipocytes in said population are Wnt⁺ adiopocytes. In some embodiments, said subject in need thereof has type 1 diabetes mellitus. In some embodiments, the subject in need thereof has type 2 diabetes mellitus. In some embodiments, the subject in need thereof has hepatic steatosis. In some embodiments, the subject in need thereof has metabolic syndrome. In some embodiments, the population of exosomes is in a pharmaceutically acceptable carrier.

In another group of embodiments, the invention provides methods of ameliorating symptoms of lack of insulin production or of insulin resistance in a subject in need thereof comprising administering to the subject exosomes secreted by a population of adipocytes, at least 50% of which are Wnt⁺ adiopocytes. In some of these embodiments, at least 75% of the population of adipocytes are Wnt⁺ adiopocytes. In some of these embodiments, 90% or more of the population of adipocytes are Wnt⁺ adiopocytes. In some of these embodiments, subject in need thereof has type 1 diabetes mellitus. In some of these embodiments, the subject in need thereof has type 2 diabetes mellitus. In some of these embodiments, the exosomes are in a pharmaceutically acceptable carrier.

In another group of embodiments, the invention provides compositions comprising exosomes derived from a population of adipocytes, of which at least 50% of said population of adipocytes are Wnt⁺ adiopocytes. In some embodiments, at least 75% of said population of adipocytes are Wnt⁺ adiopocytes. In some embodiments, at least 90% of said population of adipocytes are Wnt⁺ adiopocytes. In some embodiments, the composition further comprises a pharmaceutically acceptable carrier. In some embodiments, the composition further comprises an excipient.

In still another group of embodiments, the invention provides methods for generating from human stem cells or stromal cells a population of adipocytes in which Wnt⁺ adiopocytes are present in a desired higher percentage than in naturally-occurring populations of adipocytes. The methods comprise: (a) introducing a Wnt/β-catenin reporter genetic construct into human stem cells or stromal cells of interest, which reporter genetic construct provides a detectable signal when Wnt/β-catenin signaling is present in said human stem cells or stromal cells, or in cells differentiated into adipocytes from said human stem cells or stromal cells, (b) creating duplicate first and second cultures of said human stem cells or stromal cells of interest carrying said Wnt/β-catenin reporter genetic construct, (c) subjecting said first culture of said human stem cells or stromal cells of interest carrying said Wnt/β-catenin reporter genetic construct to adipogenic induction, (d) examining differentiated adipocytes in said first culture of said human stem cells or stromal cells subjected to said adipogenic induction for the presence of said detectable signal from said Wnt/β-catenin reporter genetic construct, (e) expanding cells of said second culture of human stem cells or stromal cells if a detectable signal is detected in said first culture, and, (f) repeating steps (c)-(e) until a population of adipocytes results which contains said population of adipocytes of which said desired higher percentage of Wnt⁺ adipocytes is present than in naturally-occurring populations of adipocytes, thereby generating a population of adipocytes in which Wnt⁺ adiopocytes are present in a desired higher percentage than in naturally-occurring populations of adipocytes. In some embodiments, the human stem cells or stromal cells of interest are bone marrow-derived stromal cells, adipose-derived stem cells, or umbilical cord stem cells. In some embodiments, the human stem cells or stromal cells of interest are bone marrow-derived stromal cells. In some embodiments, the Wnt/β-catenin reporter genetic construct is Tcf.Lef:H2B-GFP or in which said sequence encoding GFP has been replaced by a sequence encoding red fluorescent protein or yellow fluorescent protein. In some embodiments, the desired higher percentage in which of Wnt⁺ adiopocytes are present than in naturally-occurring populations of adipocytes is 50%. In some embodiments, the desired higher percentage in which of Wnt⁺ adiopocytes are present than in naturally-occurring populations of adipocytes is 75%. In some embodiments, the desired higher percentage in which of Wnt⁺ adiopocytes are present than in naturally-occurring populations of adipocytes is 90%. In some embodiments, the desired higher percentage in which of Wnt⁺ adiopocytes are present than in naturally-occurring populations of adipocytes is 95% or higher. In some embodiments, the Wnt/β-catenin reporter genetic construct is introduced into said human stem cells or into said stromal cells by transfection of said cells with a viral vector. In some embodiments, the transfection of said cells with a viral vector is by a lentiviral vector. In some embodiments, the transfection of said cells with a viral vector is by an adeno-associated viral vector. In some embodiments, the Wnt/β-catenin reporter genetic construct is introduced into said human stem cells or said stromal cells by CRISPR. In some embodiments, CRISPR is used to introduce said Wnt/β-catenin reporter genetic construct into said human stem cells or said stromal cells at Rosa26.

In another group of embodiments, the invention provides methods of ameliorating symptoms of insulin production or of insulin resistance in a subject in need thereof, said method comprising administering to said subject a composition comprising a population of adipocytes, at least 50% of said population are Wnt⁺ adiopocytes. In some embodiments, at least 75% of said population of adipocytes are Wnt⁺ adiopocytes. In some embodiments, at least 95% of said population of adipocytes are Wnt⁺ adiopocytes. In some embodiments, the subject has type 1 diabetes mellitus, and said population of adipocytes is co-administered with insulin. In some embodiments, the subject in need thereof has type 2 diabetes mellitus. In some embodiments, the population of adipocytes is in a pharmaceutically acceptable carrier

### BRIEF DESCRIPTION OF THE DRAWINGS

FIGS. 1A-1F. FIG. 1A. FIG. 1A presents immunofluorescent and microscopy images of active Wnt/β-catenin signaling in vivo, indicated by green fluorescent protein (“GFP”) expression from the TCF/Lef:H2B-GFP allele, in adipocytes marked by Perilipin. Nuclei were stained with DAPI. Scale bars, 50 μm; close-up scale bars, 20 μm. FIG. 1B. FIG. 1B presents immunofluorescent and microscopy images of active Wnt/β-catenin signaling in vitro in cells labeled by GFO, BODIPY, and DAPI. Scale bars, 50 μm; close-up scale bars, 20 μm. FIG. 1C. FIG. 1C presents a graph showing the quantification of Wnt⁺ adipocytes among total adipocytes in various fat depots of adult male mice testedin the in vivo study presented in FIG. 1A. n=6-9 mice. FIG. 1D. FIG. 1D presents a graph showing the quantification of Wnt⁺ adipocytes among total adipocytes induced from adipose stromal vascular fraction (“SVF”) cells derived from adult male fat depots and bone marrow stroma in the study described in FIG. 1B. n=7-10 independent experiments. FIG. 1E. FIG. 1E presents photographs of immunofluorescent staining of Pparγ and adiponectin in cultured GFP adipocytes derived from bone marrow in the study described in FIG. 1B. n=3 independent experiments. Scale bars, 20 μm. FIG. 1F. FIG. 1F presents representative images of GFP (gray arrows) and GFP⁻ (white arrows) adipocytes induced from human bone marrow stromal cells infected with TCF/Lef:H2B-GFP reporter lentiviral virus. n=2 independent experiments, 3 independent wells each. Scale bar, 20 μm. Data are mean±s.e.m., ***P<0.001, one-way ANOVA followed by Tukey's test.

FIGS. 2A-2H. FIG. 2A. FIG. 2A presents real-time monitoring microscopy images of Wnt⁺ adipocytes during adipogenesis. The adipocytes were induced from BMSCs of T/L-GFP mice. n=3 independent experiments. Scale bar, 50 μm. FIG. 2B. FIG. 2B presents a schematic diagram of the experiments and microscopy images of Wnt⁺ and Wnt⁻ adipocytes separated by FACS. n=3 independent experiments. Scale bars, 100 μm. FIG. 2C. FIG. 2C presents time-lapse microscopy images of induced Wnt⁺ adipocytes from BMSCs of T/L-GFP mice with Ctnnb1 and control siRNA-mediated knockdown. n=2 independent experiments, 3 independent wells each. Scale bars, 50 μm. FIG. 2D. FIG. 2D presents microscopy images and Oil Red O staining of Wnt⁺ adipocytes induced from iBAT-derived SVF cells of T/L-GFP mice with control and LF3 treatment in different doses. n=5 independent experiments. Scale bars, 50 μm. FIG. 2E. FIG. 2E shows immunofluorescent images of Wnt⁺ adipocytes induced from two independent cell lines (GFPpos-1 and -2) after treatment with 50 μM LF3 for one day. LF3 was added into the medium after 3-days of pro-adipogenic induction. After 1-day of LF3 administration, GFP signals are significantly quenched in Wnt⁺ adipocytes, along with obviously reduced cell numbers. FIG. 2F. FIG. 2F shows immunofluorescent images of Wnt⁺ adipocytes induced from two independent cell lines (GFPpos-1 and -2) after treatment with 50 μM LF3 for two days. Remarkable cell death of Wnt⁺ adipocytes is seen compared to controls. Scale bar, 100 μm. FIG. 2G. FIG. 2G presents a schematic diagram of rescue experiment and microscopy images of Oil Red O staining in adipocytes induced from GFPneg-1 cell line with LF3 treatment. n=3 independent experiments. Scale bar, 50 μm. FIG. 2H. FIG. 2H presents a quantitative RT-PCR analysis of the expression of adipogenic genes in the experiment shown in FIG. 2G. The levels of mRNA expression are normalized to that of 36B4. Data are mean±s.e.m., *P<0.05, **P<0.01; ***P<0.001, n.s., not significant, one-way ANOVA followed by Tukey's test.

FIGS. 3A-3J. FIG. 3A. FIG. 3A presents a schematic diagram of scRNA-seq and scATAC-seq experiments on SVF-induced adipocytes. FIG. 3B. FIG. 3B presents a Uniform Manifold Approximation and Projection (“UMAP”) visualization of 2,537 adipocytes from iBAT (1,710 GFP⁺ and 827 GFP⁻) and 1,345 adipocytes from iWAT (984 GFP⁺ and 361 GFP⁻) in scRNA-seq. FIG. 3C. FIG. 3C presents a UMAP visualization of 4,302 adipocytes from iBAT (1,727 GFP⁺ and 2,575 GFP⁻) and 1,383 adipocytes from iWAT (562 GFP⁺ and 821 GFP⁻) in scATAC-seq. FIG. 3D. FIG. 3D presents a heat map of the expression of the top 30 signature genes in iBAT-derived Wnt⁺ (“GFPpos”) and Wnt⁻ (“GFPneg”) adipocytes in scRNA-seq. FIG. 3E. FIG. 3E presents a heat map of the expression of the top 30 signature genes in iWAT-derived Wnt⁺ (“GFPpos”) and Wnt⁻ (“GFPneg”) adipocytes in scRNA-seq. FIG. 3F. FIG. 3F presents “violin plots” of induced Wnt⁺ and Wnt⁻ adipocytes, showing the distribution of normalized expression values of some representative genes in scRNA-seq. FIG. 3G. FIG. 3G presents a hallmark gene set analysis of DEGs enriched in iWAT-derived Wnt⁺ adipocytes in scRNA-seq. FIG. 3H. FIG. 3H presents a KEGG pathway gene set analysis of DEGs enriched in iWAT-derived Wnt⁺ adipocytes in scRNA-seq. FIG. 3I. FIG. 3I presents a Wikipathway gene set analysis of DEGs enriched in iWAT-derived Wnt⁺ adipocytes in scRNA-seq. FIG. 3J. FIG. 3J presents a gene ontology term gene set analysis of DEGs enriched in iWAT-derived Wnt⁺ adipocytes in scRNA-seq.

FIGS. 4A-4H. FIG. 4A. FIG. 4A is an image of a Western blot analysis showing protein levels of AKT, phosphorylated AKT, and β-actin in adipocytes induced from mBaSVF, GFPpos-1, and GFPneg-1 cell lines. n=3 independent experiments. FIG. 4B. FIG. 4B is an image of a Western blot analysis showing protein levels of GSK-3β, phosphorylated GSK-3β, 4E-BP1, and phosphorylated 4E-BP1 in adipocytes induced from mBaSVF, GFPpos-1, and GFPneg-1 cell lines. n=3 independent experiments. FIG. 4C. FIG. 4C presents immunofluorescent images of Wnt⁺ and Wnt⁻ adipocytes induced from two independent precursor cell lines (GFPpos-1 and -2) with treatment with LY294002 (14 μM) for one day. LY294002 was added into the medium after 3 days of pro-adipogenic induction. n=5 independent experiments. Scale bar, 100 μm. FIG. 4D. FIG. 4D presents immunofluorescence images of Wnt⁺ and Wnt⁻ adipocytes induced from two independent precursor cell lines (GFPpos-1 and -2) with treatment with LY294002 (14 μM) for two days. LY294002 was added into the medium after 3 days of pro-adipogenic induction. LY294002 treatment diminished GFP signals prior to causing marked cell death in Wnt⁺ adipocytes. n=5 independent experiments. Scale bar, 100 μm. FIG. 4E. FIG. 4E presents immunofluorescence images of iWAT and iBAT of T/L-GFP mice treated with Temsirolimus (Tem) or without such treatment (Control). n=5 mice. Scale bar, 100 μm. FIG. 4F. FIG. 4F is a graph quantitating the Wnt⁺ adipocytes among the total adipocytes present in the cells shown in FIG. 4E. FIG. 4G. FIG. 4G presents immunofluorescence images of iWAT and iBAT of male Ins2Akita; T/L-GFP mice (“Akita”) or littermate controls (Control) at 8 weeks of age. n=5 mice. Scale bar, 100 μm. FIG. 4H. FIG. 4H presents graphs quantitating the number of Wnt⁺ adipocytes among the total adipocytes shown in FIG. 4G. Data are mean±s.e.m., ***P<0.001, unpaired Student's t-test.

FIGS. 5A-5I. FIG. 5A. FIG. 5A is an image of immunofluorescence staining of mitochondrial membrane potentials in Wnt⁺ adipocytes induced from iBAT-derived SVF cells of T/L-GFP mice. n=5 independent experiments. Scale bar, 100 μm; close-up scale bar, 20 μm. FIG. 5B. FIG. 5B is a graph quantitating the staining shown in FIG. 5A. AOD, average optical density. Data are mean±s.e.m., unpaired Student's t-test; ***P<0.001. FIG. 5C. FIG. 5C is a graph of the cellular oxygen consumption rate (“OCR”) of four groups of adipocytes differentiated from GFPpos-1, GFPpos-2, GFPneg-1, and mBaSVF cell lines, respectively. n=3 independent experiments. FIG. 5D. FIG. 5D presents a schematic diagram of the experimental timeline for testing the response of mice to a cold temperature, and Immunofluorescent staining of iWAT from T/L-GFP mice with 4-day thermal challenge showing close association of Wnt⁺ adipocytes with UCP1+ beige adipocytes. n=5 mice. Scale bar, 100 μm. FIG. 5E. FIG. 5E presents a schematic diagram of the experimental timeline for testing the response of mice to cold following tamoxifen administration and washout, and images of immunofluorescence-stained iWAT from tamoxifen-treated Tcf/Lef-CreERT2;Rosa26R^{mTmG} mice after 4-day cold exposure. n=8 mice. Scale bar, 50 μm. FIG. 5F. FIG. 5F presents a schematic diagram of the experimental timeline for treating T/L-DTA mice and control (Fabp4-Flex-DTA) mice with tamoxifen, resting them for 48 hours, and then exposing them to cold for 4 days, and images of immunofluorescence-stained iWAT from those mice. n=7 mice. Scale bar, 50 μm. FIG. 5G. FIG. 5G presents a graph depicting the quantitative RT-PCR analysis of gene expression in iWAT from the control (Fabp4-Flex-DTA) and T/L-DTA mice in the experiment described in FIG. 5F. n=6 (control) and 7 (T/L-DTA mice). Levels of mRNA expression are normalized to that of Adipoq. FIG. 5H. FIG. 5H is an image of a Western blot showing protein levels of UCP1, OXPHOS complexes, and β-actin in iWAT from tamoxifen-treated controls and T/L-DTA mice under cold (6° C.) and ambient (22° C.) temperatures. n=4 mice. FIG. 5I. FIG. 5I presents photographs iWAT from control and from Akita mice immunofluorescently stained for UCP1 and Perilipin. Scale bars, 50 μm.

FIGS. 6A-6E. FIG. 6A. FIG. 6A presents two graphs. The left-hand graph shows a glucose tolerance test (“GTT”) in tamoxifen-treated control (Fabp4-Flex-DTA) mice and in T/L-DTA mice on regular chow diet. n=7 mice each. The right-hand graph shows the calculated area under the curve (AUC) for the data from the glucose tolerance test. Data are mean±s.e.m., *P<0.05, **P<0.01, two-way repeated ANOVA followed by Bonferroni's test. AUC was analyzed by two-tailed t-test, P=0.0012. FIG. 6B. FIG. 6B shows a schematic diagram Wnt⁺ adipocyte gain-of-function studies by cell implantation. FIG. 6C. FIG. 6C A shows photographs of fat pad formed by implanted cells. Blue agarose beads were included to locate the Matrigel pad. The black arrow shows benign vascularization of fat pad within two weeks. Scale bar, 50 μm. FIG. 6D. FIG. 6D is a photograph showing immunofluorescent staining for Perilipin showing mature adipocytes and accompanied agarose beads (marked by B) in the ectopically formed fat pad shown in FIG. 6C. Scale bar, 50 μm. FIG. 6E. FIG. 6E shows two graphs. The left-hand graph shows the results of a glucose tolerance test (“GTT”) in mice that received implantation of committed pre-adipocytes/adipocytes from mBaSVF (n=8 mice) or GFPpos-1 (n=7 mice) cell lines for two weeks. Data are mean±s.e.m., *P<0.05, **P<0.01, two-way repeated ANOVA followed by Bonferroni's test. AUC was analyzed by two-tailed t-test, P=0.0017.

FIGS. 7A-7E. FIG. 7A. FIG. 7A presents transmission electron microscopy (TEM) images of Wnt⁺ and Wnt⁻ adipocyte exosomes induced from immortalized precursor cell lines. FIG. 7B. FIG. 7B is a photograph of a Western blot showing that exosomes isolated from Wnt⁺ adipocytes (“GFPpos”) and from Wnt⁻ adipocytes (“GFPneg”) were positive for marker CD9 and negative for GM130. FIG. 7C. FIG. 7C presents immunofluorescent staining images showed that exosomes were present within liver, epididymal white adipose tissue (“eWAT”), inguinal white adipose tissue (“iWAT”), or muscle, of mice treated with exosomes (“EVs”) secreted by Wnt⁺ adipocytes (“GFPpos”) or by Wnt⁻ adipocytes (“GFPneg”). FIG. 7D. FIG. 7D is a graph of blood glucose levels in mg/dl of Akita mice treated with exosomes (“EVs”) secreted by Wnt⁺ adipocytes (“GFPpos”). The Akita mice showed improvement of fasting blood glucose levels 24 hours after treatment. n=9 mice. FIG. 7E. FIG. 7E is a graph of blood glucose levels in mg/dl of Akita mice treated with exosomes (“EVs”) secreted by Wnt⁻ adipocytes (“GFPneg”). Akita mice treated with Wnt⁻ adipocyte EVs exhibit no improvement in fasting blood glucose levels 24 hours after treatment. n=8 mice.

FIGS. 8A-8D. FIGS. 8A-8D relate to mass spectrometry (MS) analysis of exosomes from secreted by Wnt⁺ adipocytes or by Wnt⁻ adipocytes. FIG. 8A. FIG. 8A is a gene ontology (GO) analysis showed the most enriched descriptors of thermogenesis-regulatory functions of proteins detected by MS of exosomes from Wnt⁺ adipocytes compared to those from Wnt⁻ adipocytes, indicating a paracrine function of Wnt⁺ adipocytes on beige fat biogenesis that is beneficial for whole-body glucose metabolism. FIG. 8B. FIG. 8B is a table showing that adipsin (encoded by Cfd), an adipocytokine in the bloodstream that improves pancreatic β cell function, was found to be significantly enriched in exosomes secreted by Wnt⁺ adipocytes. FIG. 8C. FIG. 8C presents graphs of single-cell RNA-seq in interscapular BAT (“iBAT”) SVF-induced adipocytes and inguinal white adipose tissue (“iWAT”) SVF-induced adipocytes, bulk RNA-seq, and quantitative PCR data confirming that Cfd (which encodes adipsin) is preferentially expressed in Wnt⁺ adipocytes (“GFPpos”) compared its expression in Wnt⁻ adipocytes (“GFPneg”). FIG. 8D. FIG. 8D presents the results of an ELISA presenting the concentration of adipsin, in pg/μl, in T/LDTA mice whose Wnt⁺ adipocytes were ablated. n=6 DTA mice, 5 controls.

FIG. 9. FIG. 9 is a table presenting the most highly enriched genes generated using MS to estimate the functional enrichment and biological pathway of input Wnt⁺ adipocyte exosomes based on the differential gene expression compared to those secreted by Wnt⁻ adipocytes. The left column provides the common abbreviation for the gene (such as Cfd, the gene encoding adipsin). The next three columns show selected KEGG, Wild, and Reactome pathway analyses for the genes. The column labeled “Wnt⁺/Wnt⁻” shows the relative gene expression of the gene in exosomes secreted by Wnt⁺ adipocytes compared to exosomes secreted by Wnt⁻ adipocytes. The level of Cfd, which encodes adipsin, for example, is shown to be ˜3.7 times higher in Wnt⁺ adipocyte exosomes compared to exosomes secreted by Wnt⁻ adipocytes. The final two columns show the relative abundances for each group calculated by the bioinformatics program.

### DETAILED DESCRIPTION

### Introduction

Canonical Wnt signaling is known to be required during the differentiation of osteocytes from murine bone marrow stromal cells (“BMSCs”). Studies of osteocyte differentiation were undertaken using BMSCs from the well-defined Wnt/β-catenin signaling specific reporter mouse line TCF/Lef:H2B-GFP (hereafter “T/L-GFP”) that allows for real-time monitoring of Wnt/β-catenin activity at single-cell resolution. As a negative control, adipocytes were differentiated from the T/L-GFP BMSCs in parallel to the osteocytes, as adipocytes were not expected to display the activation of Wnt/β-catenin signaling. Surprisingly, the presence of a small population of green fluorescent protein (“GFP”)-positive cells containing lipid droplets was found. The cells were originally thought to be artifacts or not to be adipocytes, but the cells were observed repeatedly and found to be properly characterized as adipocytes. This surprising and unexpected observation prompted the conduct of a careful survey for the presence of active Wnt/β-catenin signaling in any developing and mature adipocytes of fat depots from T/L-GFP mice.

In some embodiments, the present invention therefore relates to the surprising discovery of a previously unknown population of adipocytes marked by active intracellular Wnt/β-catenin signaling. Adipocytes marked by this signaling are usually referred to herein as “Wnt⁺ adipocytes.” As adipocytes marked by this signaling were not previously known to exist, the studies reported herein elucidate for the first time the role of Wnt⁺ adipocytes in converting white fat cells to beige cells and their role in thermogenesis.

Studies were conducted examining varying murine fat depots, including both white and brown adipose tissues, to determine how much of the various adipose tissues studied were composed of Wnt⁺ adipocytes. The percentage of total murine fat cells that were found to be Wnt⁺ ranged from under 2% in epididymal white adipose tissue (“eWAT”) to approximately 20% in interscapular brown adipose tissue (“iBAT”), with the fat depots of male mice containing higher percentages of Wnt⁺ adipocytes than those of females, and the fat depots of younger adult mice containing higher percentages of Wnt⁺ adipocytes than the fat depots of old mice. Thus, the highest percentage of Wnt⁺ adipocytes in any fat depot studied to date is approximately 20%. In contrast, the methods reported in the Examples below, provided the production of populations of murine and human adipocytes in which 95% or more of the adipocytes are Wnt⁺.

Therefore, in some aspects, the invention provides methods allowing the production of populations of adipocytes that are almost entirely composed of Wnt⁺ adipocytes and result in percentages of Wnt⁺ adipocytes that do not appear to exist in natural fat depots. For example, the populations of adipocytes produced by the methods disclosed herein can be composed of adipocytes of which 30% or more are Wnt⁺, 40% are Wnt⁺, 50% are Wnt⁺, 60% are Wnt⁺, 70% are Wnt⁺, 80% are Wnt⁺, 90% are Wnt⁺, or 91%, 92%, 93%, 94%, 95%, 96% 97%, 98%, 99% or 100% are Wnt⁺, with each successively stated higher percentage of Wnt⁺ adipocytes being preferred to each previously stated lower percentage. Accordingly, the populations of adipocytes produced by the methods described herein are expected to be surprisingly more potent and surprisingly more useful than any previously described population of adipocytes, which would contain only a naturally occurring percentage of Wnt⁺ adipocytes.

It should also be noted that, since Wnt⁺ adipocytes were not previously known to exist, there was no method available in the art prior to the present disclosure to determine which, if any Wnt⁺ adipocytes are present in a naturally-occurring population of adipocytes, to preferentially create a population enriched in Wnt⁺ adipocytes compared to the low percentages of such cells present in naturally-occurring percentages of adipocytes, or to separate Wnt⁺ adipocytes from Wnt⁻ adipocytes to create a population of adipocytes with a higher percentage of Wnt⁺ adipocytes than that of the starting, naturally-occurring population.

In a further embodiment, the present disclosure reports the surprising discovery that the cells of this previously unknown type of adipocytes had beneficial effects on blood glucose control both in mice that received implantations of extra Wnt⁺ adipocytes and in mice that lacked Wnt⁺ adipocytes. A murine model lacking Wnt⁺ adipocytes showed significantly impaired glucose tolerance, which was dramatically improved when Wnt⁺ adipocytes were implanted. The findings regarding the effect of Wnt⁺ adipocytes on glucose metabolism indicate that the cells will be useful in ameliorating the effect of insulin resistance in mammals with type 2 diabetes mellitus (“T2DM”).

In another embodiment, the present disclosure reports the surprising discovery that extracellular vesicles, including exosomes, produced by Wnt⁺ adipocytes had beneficial impacts on blood glucose control in Akita mice, a recognized murine model of type 1 diabetes mellitus (“T1DM”). Animals receiving injections of extracellular vesicles from Wnt⁺ adipocytes exhibited dramatically reduced blood glucose levels as compared to animals receiving exosomes secreted by Wnt⁻ adipocytes. These in vivo studies show that exosomes secreted by Wnt⁺ adipocytes will be useful in ameliorating the effects of reduced insulin production in mammals with T1DM.

In another aspect of the invention, the studies reported below show that this previously unknown population of adipocytes produce exosomes that contain surprising levels of compounds, such as proteins, beneficial to controlling glucose metabolism in animals. The levels of these compounds in exosomes, derived from Wnt⁺ adipocytes were found to be multiples of the levels of the same compounds in exosomes, derived from Wnt⁻ adipocytes. For example, the adipokine adipsin has been shown to preserve pancreatic beta cell function and to ameliorate hyperglycemia in diabetic mice, while higher levels of circulating adipsin are associated with a significantly lower risk of developing T2DM in adult humans. See, e.g., Gomez-Banoy, et al., Nat Med, 2019, 25:1739-1747, doi:10.1038/s41591-019-0610-4. Studies reported below that compared proteomics in the exosomes derived from Wnt⁺ adipocytes against those derived from Wnt⁻ adipocytes showed that exosomes secreted by Wnt⁺ adipocytes contained levels of adipsin almost four times that of exosomes secreted by Wnt⁻ adipocytes. Similar surprisingly elevated levels of other compounds, such as proteins, important to metabolism were also seen. Thus, the exosomes produced by Wnt⁺ adipocytes are far more potent sources of proteins and factors beneficial for protecting pancreatic beta cells, for protecting adults from developing T2DM, and for regulating glucose metabolism in T1DM, than are the exosomes secreted by Wnt⁻ adipocytes.

As noted above, the fat depots of tissues examined thus far had populations of adipocytes in which the percentage of Wnt⁺ cells ranged from about 2% to about 20%. It is expected that the relatively small population of Wnt⁺ adipocytes in natural fat depots, or produced by differentiated adipocytes from, for example, adipose tissue-derived stem cells, by previous techniques for generating adipocytes, would produce a correspondingly small percentage of exosomes from Wnt⁺ cells, dispersed within the much higher percentage of exosomes from the predominating Wnt⁻ cells. In contrast, the present invention provides the ability to produce populations of adipocytes highly enriched in all Wnt⁺ adipocytes compared to naturally-occurring populations. In studies underlying the present disclosure, populations of adipocytes were produced in which over 95% were Wnt⁺ adipocytes. The population of exosomes from cloned/enriched Wnt⁺ cells are expected to be correspondingly more enriched in factors, such as adipsin, affecting glucose metabolism and regulation, and correspondingly more potent, than compositions using exosomes derived from any adipocyte population produced using methods available prior to the discoveries reported in this disclosure. Accordingly, the populations of exosomes produced by the methods described herein are expected to be surprisingly more potent and surprisingly more useful than previously described adipocyte-derived exosomes.

Adipocytes, Wnt⁺ Adipocytes, and Methods of Making Wnt⁺ Adipocytes

Adipose tissue, or fat, was originally understood to be merely a depository for lipid molecules. More recently, adipose tissue has become recognized as a tissue critical as a “regulator of energy balance and nutritional homeostasis,” containing white adipocytes, that make up the bulk of fat tissue, brown adipocytes, that generate heat through the action of uncoupling protein-1 (UCP-1), a protein located in the mitochondria, and, in rodents, inducible brown fat-like cells called “beige” or “brite” cells. (See, Rosen and Spiegelman, Cell, 2014, 156(1-2):20-44, doi.org/10.1016,j.cell.2013.12.012). Adipocytes have been determined to communicate with other fat cells and with other cell types by a number of autocrine, paracrine, and endocrine agents, collectively known as adipokines. See, Rosen and Spiegelman, supra. These include the complement factor adipsin, the hormone leptin, and the adipokine adiponectin, which serves as a biomarker for insulin resistance and metabolic dysfunction, among many others. Id.

Adipokines have effects on more than just fat deposits. The adipokine adipsin, in particular, has been shown to improve pancreatic β cell function. Replenishment of adipsin in diabetic mice was shown to treat hyperglycemia by boosting insulin secretion. See, Lo, et al., Cell, 2014, 158:41-53, doi:10.1016/j.cell.2014.06.005. Another study reported that chronic replenishment of adipsin in diabetic db/db mice ameliorated hyperglycemia and increased insulin levels, while preserving beta cells, and that middle-aged adult humans with higher circulating levels of adipsin were at lower risk of developing diabetes. See, Gomez-Banoy, et al., Nat Med, 2019, 25:1739-1747, doi:10.1038/s41591-019-0610-4. The studies reported in the Examples, below, indicate that exosomes from Wnt⁺ adipocytes are highly enriched in adipsin and other beneficial metabolic factors compared to those from Wnt⁻ adipocytes. In combination with the studies reported by Lo et al., supra, and by Gomez-Banoy et al., supra, the studies reported in the Examples indicate that Wnt⁺ adipocytes and exosomes from Wnt⁺ adipocytes will be particularly useful as agents to ameliorate hyperglycemia, to increase insulin levels, and to reduce the tendency of individuals to develop diabetes.

The Examples show the induction and isolation of Wnt⁺ adipocytes from both murine and human stromal cells. Cells were subjected to initial fluorescence activated cell sorting, or “FACS,” sorting to exclude endothelial cells, blood cells, and lymphocytes, using antibodies that recognized cell surface markers (e.g., CD31, CD45, and TER119) on such cells. Human bone marrow stromal cells were transfected with a gene construct, “Tcf.Lef:H2B-GFP,” known to allow detection of Wnt/β-catenin signaling. The Tcf.Lef:H2B-GFP construct was disclosed in 2010 (see, Ferrer-Vaquer, et al., BMC Dev Biol., 2010, 10:121, doi:10.1186/1471-213X-10-121) and is available for purchase as a plasmid from Addgene (Watertown, MA, catalog no. 32610). The plasmid contains a sequence encoding green fluorescent protein (“GFP”), and detection of GFP by fluorescent imaging of cells indicates that the cells glowing green are Wnt⁺ adipocytes.

While GFP is a convenient label for use with FACS or other flow cytometric techniques, other detectable labels are known and can be used in place of GFP as a marker to identify Wnt⁺ adipocytes. Referring only to fluorescent markers, for example, genetic sequences encoding Red Fluorescent Protein (“RFP”) or Yellow Fluorescent Protein (“YFP”) could be substituted for the one encoding GFP in the gene construct.

In the studies reported in the Examples below, lentivirus was used to transfect human bone marrow stromal cells to introduce a gene construct bearing the detectable label. Other viruses are, however, known to transfect human cells, and can be used in place of the lentivirus discussed in the studies below to introduce a reporter gene into human stem cells or stromal cells to allow detection of Wnt/β-catenin signaling in adipocytes when the transfected stem cells are then differentiated into adipocytes. For example, a number of adeno-associated viruses (“AAVs,” see, e.g., Naso et al., BioDrugs, 2017, 31(4):317-334, doi:10.1007/s40259-017-0234-5) are being tested as vectors for gene therapy in human clinical trials (see, e.g., Costa Verdera, et al., Mol Ther., 2020, 28(3):723-746, doi: 10.1016/j.ymthe.2019.12.010), such as AAV6 and AAV9, and can be used instead of the lentiviral vector discussed in the Examples. Alternatively, a reporter construct can be introduced into the stem cells by CRISPR at a targeted site, such as the Rosa26 position in human MSCs. The location of Rosa26 was established in human embryonic stem cells was reported in 2007 (see, Trion, et al., Nat Biotechnol., 2007, 25(12):1477-82, doi: 10.1038/nbt1362).

As noted in the Introduction, the studies reported below were performed using human bone marrow derived stromal cells, in part because the discovery of Wnt⁺ adipocytes was an accidental finding in the course of studies intended to study osteogenesis. While bone marrow is an important source of adipocytes (see, e.g., Arner, Nestle Nutr Inst Workshop Ser, 2018, 89:37-45, doi:10.1159/000486491), adipocytes have been differentiated from other stem cell types, including human adipose-tissue derived mesenchymal stem cells (see, e.g., Bunnell et al., Methods, 2008, 45(2):115-120, doi.org/10.1016/j.ymeth.2008.03.006; Huang et al., Cell Transplantation, 2013, 22(4):701-709, doi.org/10.3727/096368912X655127; Cuaranta-Monroy, Stem Cell Res, 2014, 13(1):88-97), and reagents to induce differentiation of stem cells into adipocytes are commercially available from a number of sources, including example, iXCells Biotechnologies (San Diego, CA, product code MD-0005-250M, for adipose-derived stem cells), R&D Systems, Inc. (Minneapolis, MN, catalog no. CCM007, for mesenchymal stem cells), and Thermo Fisher Scientific (Waltham, MA, catalog no. A1007001).

One virtue of using stem cells of human origin, such as human MSCs or human umbilical cord stem cells, is the ability to establish human-originated immortalized precursor cell lines of Wnt⁺ adipocytes. Such cell lines allow Wnt⁺ adipocytes to be induced from precursor cells, with the induced Wnt⁺ adipocytes then available to secrete exosomes to be isolated for therapeutic use. Consistent with the low percentage of Wnt⁺ adipocytes present in the fat depots (e.g., white adipose tissue, brown adipose tissue, and bone marrow), the percentage of Wnt⁺ adipocyte precursor cells in isolated MSCs, whether stromal vascular fractions (“SVFs”) of adipose tissue or bone marrow mesenchymal stromal cells (“BMMSCs”), is rather low. To produce precursor cell lines of Wnt⁺ adipocytes, human stem cells or stromal cells that have been transfected with a Wnt/β-catenin reporter construct are immortalized and cultured in duplicate tissue cultures. To produce populations of adipocytes with percentages of Wnt⁺ adipocytes higher than those present in naturally-occurring populations, serial dilution cultures can be used. The following is an exemplar protocol for obtaining such populations: 1) introduce the reporter construct into the precursor stem cells of choice (such as MSCs or SVFs, 2) set up duplicate pools of cultures of the stem cells (such as the MSCs/SVFs) carrying the reporter construct, 3) subjecting one duplicate of each pool to adipogenic induction, and examining differentiated adipocytes for the presence of GFP⁺ cells (which reveals the presence of Wnt⁺ adipocytes), 4) once Wnt⁺ adipocytes are seen in one culture, its duplicate is known to contain Wnt⁺ adipocyte precursor cells and can be subjected to expansion, 5) the cultures can then be subjected to serial limited dilution cultures for selection of higher percentages of Wnt⁺ adipocyte precursor cells by repeating the methods 2-4 until a pool of precursor cells is obtained that can differentiate under adipogenic induction into a population of adipocytes with any desired percentage of Wnt⁺ adipocytes. In the studies reported in the Examples, following this protocol resulted in populations of adipocytes in which more than 95% of the adipocytes were Wnt⁺.

One of the duplicate cultures is then induced to differentiate into adipocytes. Once Wnt⁺ adipocytes are seen in one culture, its duplicate, which contains immortalized precursor cells, is subjected to expansion and then to serial limited dilution culture for selection of pools containing high percentages of Wnt⁺ adipocyte precursor cells (once again determined by the presence of Wnt⁺ adipocytes in a duplicate subjected to adipogenic induction). With several rounds of repeated selection, precursor cell lines for Wnt⁺ can be isolated and established. If desired, other cultures, of duplicate Wnt⁻ adipocytes can be isolated and established, to serve as controls for or comparisons to the Wnt⁺ adipocytes.

A precursor cell line that, once subjected to adipogenic induction, can differentiate into a population with a desired percentage of Wnt⁺ adipocytes, is considered a “Wnt⁺ adipocyte precursor cell line”. In some embodiments, the Wnt⁺ adipocyte precursor cell line is differentiated into a population of adipocytes of which at least 30% are Wnt⁺ adipocytes. In some embodiments, the Wnt⁺ adipocyte precursor cell line is differentiated into a population of adipocytes of which at least 40%, 50%, 60%, 70%, 80%, 90%, 95%, 96%, 97%, 98%, or 99% are Wnt⁺ adipocytes, with each successive higher percentage being to any preceding lower percentage.

### Extracellular Vesicles and Exosomes

As described in a review by Doyle and Wang (Cells, 2019, 8(7):727; doi: 10.3390/cells8070727, incorporated herein by reference), extracellular vesicles (sometimes abbreviated herein as “EVs”) are lipid bound vesicles secreted by cells into the extracellular space. Doyle and Wang characterize EVs as comprising three main subtypes, microvesicles (“MVs”), exosomes, and apoptotic bodies, differentiated by their “biogenesis, release pathways, size, content, and function” A 2020 review by Kalluri and LeBleu, (Science, 2020, 367(6478):eaau6977, doi:10.1126/science.aau6977, hereafter referred to as “Kalluri and LeBleu” and incorporated herein by reference), states that EVs are broadly divided into ectosomes and exosomes, with ectosomes, which they state “include microvesicles, microparticles, and large vesicles in the size range of ˜50 nm to 1 μm in diameter,” formed by being pinched off from the plasma membrane, while exosomes are EVs of “˜40 nm to 160 nm in diameter (average ˜100 nm) in diameter with an endosomal origin.”

A number of references explain the biogenesis of such vesicles, how to classify them, and how to use them, including: Thery et al., J Extracell Vesicles, 2018, 7; doi.org/10.1080/20013078.2018.1535750; Kowal et al., Proc Natl Acad Sci USA, 2016, 113(8):E968-E977, doi.org/10.1073/pnas.1521230113, Woith, et al., Int J Mol Sci. 2019; 20(22):5695, doi.org/10.3390/1jms20225695; Van Niel, et al., Nat Rev Mol Cell Biol, 2018, 2018:213-28, doi.org/10.1038/nrm.2017.125; Jadli, et al., Mol Cell Biochem, 2020, 467:77-94, doi.org/10.1007/s11010-020-03703-z; Bebelman, et al., Pharmacol Ther, 2018, 2018:1-11, doi.org/10.1016/j.pharmthera.2018.02.013; and Nikfarjam, et al., J Transl Med, 2020, 2020:449; doi.org/10.1186/s12967-020-02622-3.

Thus, the term “extracellular vesicles” refers to the genus of these vesicles, while “exosomes” refers to a subset of vesicles specifically of endosome origin. The Wnt⁺ adipocyte-derived extracellular vesicles used in the studies reported in the Examples are believed to be exosomes. That belief is supported by the fact that the methods used to isolate the vesicles are methods specifically designed to isolate exosomes and the isolation kits used are specifically intended for isolating exosomes. Electron microscopy examination of isolated EVs showed that a majority of the vesicles had a diameter of ˜100 nm and Western blot assays checking for the exosome maker CD9 found abundant CD9 in EVs from both Wnt⁺ adipocytes and Wnt⁻ adipocytes, but no or very low levels of CD9 in adipocytes. Nonetheless, the possibility of contamination by other secreted vesicles was not definitively ruled out. While it is expected that the great majority, if not all, of the vesicles present in the studies reported herein were therefore exosomes, it is possible that some other vesicles were inadvertently present as well. As large vesicles pinch off from the plasma membrane and therefore may present antigens that would provoke an immune response in subjects to which allogenic exosomes are administered, the methods used to isolate exosomes are preferably ones that result in no more than 10% of the isolated extracellular vesicles being large vesicles, and preferably result in 9%, 8% 7%, 6% 5%, 4%, 3%, 2%, 1% or zero percent of the extracellular vesicles being large vesicles, with each lower percentage being preferable to any higher percentage of such vesicles being present.

As used herein, the term “exosomes” with respect to the vesicles derived from Wnt⁺ adipocytes and Wnt⁻ adipocytes, encompasses both exosomes and any other vesicles that might incidentally accompany exosomes isolated by standard methods designed specifically to provide isolated exosomes, using reagents such as a Thermo Fisher Scientific “Total Exosome Isolation” kit. In preferred embodiments, the exosome compositions provided in these aspects of the invention consist of extracellular vesicles of which 90% or more are exosomes, such as in which 91%, 92%, 93%, 94%, 95%, 96%, 97%, 98%, 99%, or 100% are exosomes, with each successively stated higher percentage being preferred to any previously stated lower percentage.

Thirty years of research has established that exosomes, originally thought to be carriers of cellular debris, are in fact a form of intercellular communication and cross-talk that carry proteins, microRNA, long non-coding RNA, and other mediators of signaling. They are important not only in normal cellular signaling and metabolism, but also in tumor angiogenesis and growth. Years of study have resulted in multiple methods of isolating exosomes, which allow the practitioner to balance the total yield of EVs against the specificity of EVs isolated. The methods include, grouped in order of high yield but lower specificity to lower yield but higher specificity: (a) precipitation, (b) ultrafiltration, size-exclusion chromatography, and ultracentrifugation, (c) density gradient centrifugation, and (d) immunoaffinity. As these methods have been developed over years, it is assumed that persons of skill are familiar with the advantages and drawbacks of each of these methods, as well as adjustments to make to the various methods depending on the particular biofluid (e.g., plasma, serum, urine, cerebrospinal fluid) from which the EVs are to be isolated. Kits for the specific isolation of exosomes are commercially available from a number of sources, as exemplified by the “Total Exosome Isolation Kit” (catalog no. 4484450, Thermo Fisher Scientific Inc., Waltham, MA), “Exosome Isolation Kits” for cell culture media/urine and for serum/plasma, respectively (catalog nos. EIK-01 and -02, Creative Biolabs, Shirley, NY), and CD9 and CD63 immunobeads for exosome isolation (catalog nos. NBP3-11768 and NBP3-11769, respectively, Novus Biologicals, LLC, Centennial, CO).

It is also assumed that the person of skill is familiar with the various techniques that have been used to confirm the presence of exosomes after isolation, including electron microscopy, Western blot, ELISA, and flow cytometry. (See, e.g., Doyle and Wang, supra, Crescitelli, et al., Nat Protoc, 2021, 16:1548-80, doi.org/10.1038/s41596-020-00466-1; Konoshenko et al., Biomed Res Int, 2018; doi.org/10.1155/2018/8545347; Cocozza et al., Cell, 2020, 182:262-262.el, doi.org/10.1016/j.cell.2020.04.054). Accordingly, detailed information about these methods need not be set out in detail here.

Use of Wnt⁺ Adipocytes and Wnt⁺ Adipocyte-Derived Exosomes to Ameliorate Diabetes or Other Hepatic Disorders

Cells of some cell types, and exosomes from such cells, are considered as effective treatment strategies for certain diseases. He et al. (Stem Cell Research & Therapy, 2020, 11:223; doi.org/10.1186/s13287-020-01731-6) reported in 2020 that mesenchymal stem cell (“MSC”)-based therapy was considered a treatment strategy for diabetes and for hepatic disorders, such as liver cirrhosis and non-alcoholic fatty liver disease. He et al. reported that human umbilical cord MSC-derived exosomes, which they termed “HucMDEs,” promoted hepatic glycolysis, glycogen storage, and lipolysis in a Type 2 diabetes mellitus (“T2DM”) animal model. He et al. concluded that their HucMDEs “effectively alleviated hyperglycemia by improving [pancreatic] islet function, promoting glycolysis/glycogen synthesis, and inhibiting gluconeogenesis in T2DM models both in vivo and in vitro”. Similarly, Sun et al., reported in 2018 that human umbilical cord MSC-derived exosomes, which they termed “HucMSC-ex,” had a therapeutic effect in a rat T2DM model. They reported that intravenous injection of HucMSC-ex reduced blood glucose levels, partially reversed insulin resistance, improved glucose homeostasis, and relieved β-cell destruction. (See, Sun et al., ACS Nano, 2018, 12(8):7613-7628, doi: 10.1021/acsnano.7b07643.) Gesmundo et al., recently reported that EVs derived from healthy adipocytes increased survival and proliferation of pancreatic β cells and islets, while EVs derived from adipocytes from obese donors were harmful. (Gesmundo et al., JCI Insight, 2021, 6(5):e141962, doi: 10.1172/jci.insight.141962 (hereafter, “Gesmundo, et al.”). And, Nojehdehi et al. reported in 2018 that adipose tissue-derived mesenchymal stem cells injected intraperitoneally into mice with streptozotocin-induced T1DM showed an amelioration of autoimmune reaction as reflected in a significant increase in pancreatic islet cells in treated mice. See, Nojehdehi et al., J Cellular Biochem, 2018, 119(11):9433-9443, doi.org/10.1002/jcb.27260. Thus, both adipocytes and exosomes isolated from adipocytes have been shown to ameliorate symptoms or effects of T1DM and T2DM in animal models.

### Use of Wnt⁺ Adipocytes

The studies reported in the Examples, below, included pathway analyses of RNA-seq data, showing that Wnt⁺ adipocytes possess a thermogenic character and increased insulin/AKT/mTOR signaling activity compared to Wnt⁻ adipocytes. (As noted above, Wnt⁻ adipocytes were found to constitute approximately 80% to approximately 98% of the cells present in the fat depots analyzed to date). Further, some beneficial genes/proteins related to glucose metabolism, were found at a higher level in Wnt⁺ adipocytes in RNA-seq studies, compared the expression of genes related to glucose metabolism and regulation in Wnt⁻ adipocytes. For example, Wnt⁺ adipocytes had expression levels of Cdf, the gene encoding the important adipokine adipsin that were almost four times that of Wnt⁻ adipocytes. Further, animal studies in the Akita mouse model of T1DM showed that Wnt⁺ adipocytes are stronger in insulin signaling than Wnt⁻ adipocytes. Thus, Wnt⁺ adipocytes are surprisingly advantageous for protecting pancreatic beta cells, for protecting adults from developing T2DM, and for regulating glucose metabolism, that are the Wnt⁻ adipocytes which comprise the great majority of cells naturally present in an individual. Accordingly, Wnt⁺ adipocytes are expected to be more potent than are Wnt⁻ adipocytes when administered as a cellular therapeutic to ameliorate symptoms or effects of a condition related to defects in blood glucose control and regulation, such as T1DM or T2DM. Greater potency is expected to mean that fewer Wnt⁺ adipocytes need to be administered to achieve the same effect as a greater number of a standard population of adipocytes, of which 80% or more would be Wnt⁻. Fewer cells per administration can mean reduced costs in manufacturing, storing, and administering the cells, and reduce any side effects the subject might have to the cells themselves.

As noted in the Introduction, analyses of fat depots showed that the percentage of Wnt⁺ adipocytes in the depots studied to date ranged from approximately 2% to approximately 20%. And, since the existence of Wnt⁺ adipocytes was not known before the studies, there was no ability to select for or to produce populations of adipocytes that were Wnt⁺. Thus, there is no basis on which to expect that the adipocytes used as cellular therapies in the studies reported to date, including those in which adipocytes were derived from MSCs or from umbilical cord stem cells, would have contained a percentage of Wnt⁺ adipocytes higher than the ones of the fat depots studied to date. In contrast, the present disclosure not only identifies this previously unknown population of adipocytes, but also shows how to produce populations of adipocytes that have Wnt⁺ adipocytes in percentages higher than those currently known to exist in nature. In studies reported in the Examples, for example, the percentage of Wnt⁺ adipocytes in the populations of adipocytes produced by the methods taught herein was greater than 96%.

Thus, in some embodiments, the populations of adipocytes used in the methods disclosed herein can be composed of adipocytes of which 30% or more are Wnt⁺, 40% are Wnt⁺, 50% are Wnt⁺, 60% are Wnt⁺, 70% are Wnt⁺, 80% are Wnt⁺, 90% are Wnt⁺, or 91%, 92%, 93%, 94%, 95%, 96% 97%, 98%, 99% or 100% are Wnt⁺, with each successively stated higher percentage of Wnt⁺ adipocytes being preferred to each previously stated lower percentage. As noted, the percentage of Wnt⁺ adipocytes in each of these populations is higher than that of any known natural population of adipocytes. In some embodiments, the populations of adipocytes are generated from stem cells from the intended recipient, such as adipose-tissue derived stem cells or bone-marrow derived stem cells. In some embodiments, the population of adipocytes are generated from stem cells from a healthy donor. As the levels of Wnt⁺ adipocytes were found in the animal studies to be higher in males, preferably the stem cells are from a male donor. As umbilical cord stem cells are a good source of MSCs, may contain reduced major histocompatibility complex determinants, and can be differentiated into adipocytes (see, e.g., Ding, et al., Cell Transplantation, 2014, 24(3):339-347; Saben, et al., Exp. Biol. Med., 2014, 239(10):1340-51), they are a preferred source of adipocytes for some embodiments.

The studies reported in the Examples indicated that Wnt⁺ adipocytes require the presence of insulin for survival. This is not a problem in treating T2DM subjects with Wnt⁺ adipocytes, as T2DM is characterized by insulin resistance, not a lack of insulin. T1DM, however, is characterized by a reduced production of insulin. Individuals who do not have severe cases of T1DM, such as those who have only recently developed it after sufficient damage has occurred to their pancreatic islet cells to become symptomatic, are expected to produce enough insulin to sustain exogenously-sourced Wnt⁺ adipocytes. Individuals with more severe disease routinely take a form of insulin, typically either injected with each meal or in a longer-acting form. In such individuals, the Wnt⁺ adipocytes can be co-administered with the insulin so that they have insulin to support their survival for a period of time while they exert their beneficial effects.

Various studies, such as Gesmundo, et al., supra, indicate that adipocytes from persons who are obese or who have diabetes may be more harmful than beneficial. It is not clear whether such concerns would be true for adipose-derived mesenchymal stem cells or bone marrow stromal cells, as it is possible any disadvantageous factors secreted by adipocytes in obese of diabetic individuals could be due, for example, by exposure of the adipocytes to inflammatory factors after they are fully differentiated. Studies also indicate that cells from aged individuals can be problematic. Thus, when differentiating Wnt⁺ adipocytes from adipose-derived mesenchymal stem cells or from bone marrow stromal cells, it is preferable to obtain the stem or stromal cells from the adipose tissue or bone marrow of young, healthy, individuals. The adipose tissue of such individuals can be obtained, for example, from plastic surgeons performing liposuction. Bone marrow can be obtained from, for example, orthopedic surgeons or from organ donors. As umbilical cord stem cells are always from young individuals, they can be obtained from hospitals delivering babies. It is noted that primary human bone marrow stromal cells were used in the studies reported below in part because they are commercially available.

Exosomes Derived from Wnt⁺ Adipocytes

Exosomes from adipose-derived mesenchymal stem cells (“ADMSCs”) have been reported to attenuate retinal damage in a rabbit model of diabetes (Safwat et al., J Circulating Biomarkers, 2018, 7:1849454418807827, doi: 10.1177/1849454418807827), to promote healing of wounds and foot ulcers in rat models of diabetes (Shi et al., Amer J Physiology/Cell Physiology, 2020, 318(5):C848-C856, doi.org/10.1152/ajpcell.00041.2020; Li et al., Exper and Molecul Med, 2018, 50:1-14), and to ameliorate erectile dysfunction in a rat model of T2DM (Chen, F., et al., J Sexual Med, 2017, 14(9):1084-1094 and Zhu et al., Andrologia, 2017, 50(2):e12871, doi.org/10.1111/and.12871). And Zhao et al. reported in 2018 that treatment of obese mice with ADMSC-derived exosomes improved their metabolic homeostasis, including improved insulin sensitivity, reduced obesity, and alleviated hepatic steatosis. See, Zhao et al., Diabetes, 2018, 67(2):235-247, doi.org/10.2337/db17-0356.

Thus, there is extensive evidence from animal studies that exosomes derived from adipose tissue-derived MSCs can ameliorate a variety of the effects of T1DM and of T2DM and can improve the insulin sensitivity, reduce obesity, and alleviate hepatic steatosis in obese animals. Further, the Sun et al. and He et al. studies show that human umbilical cord MSC-derived exosomes also ameliorate symptoms of T2DM. Finally, the studies reported in the Examples show that exosomes from Wnt⁺ adipocytes differentiated from human bone marrow stromal cells are effective in improving glucose regulation in an animal model of T1DM. These studies together show that exosomes from adipocytes generated from three different stem cell sources ameliorate aspects of T1DM, T2DM, or both.

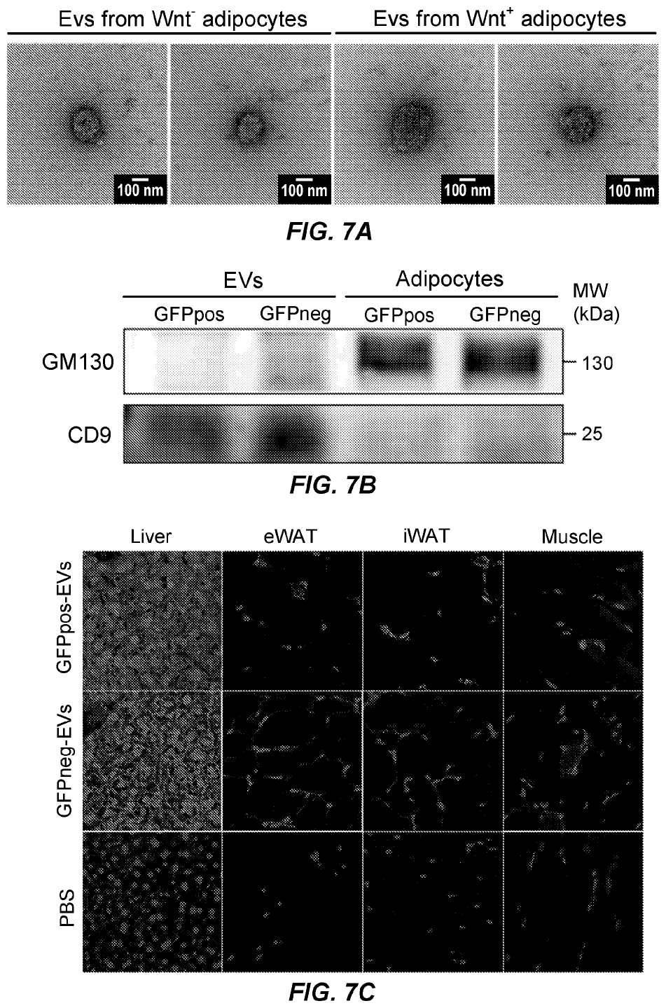

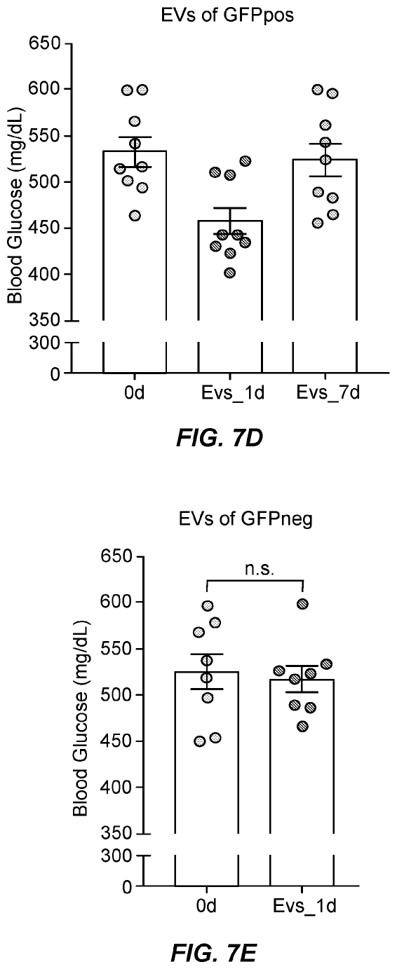

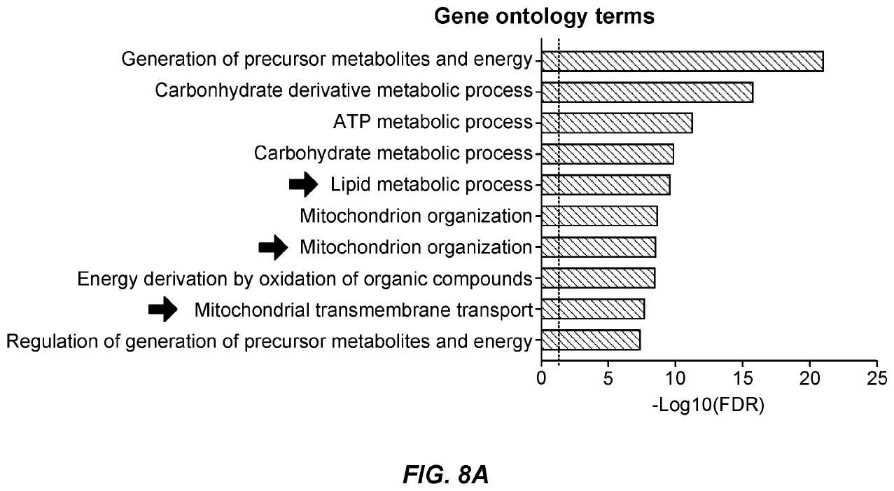

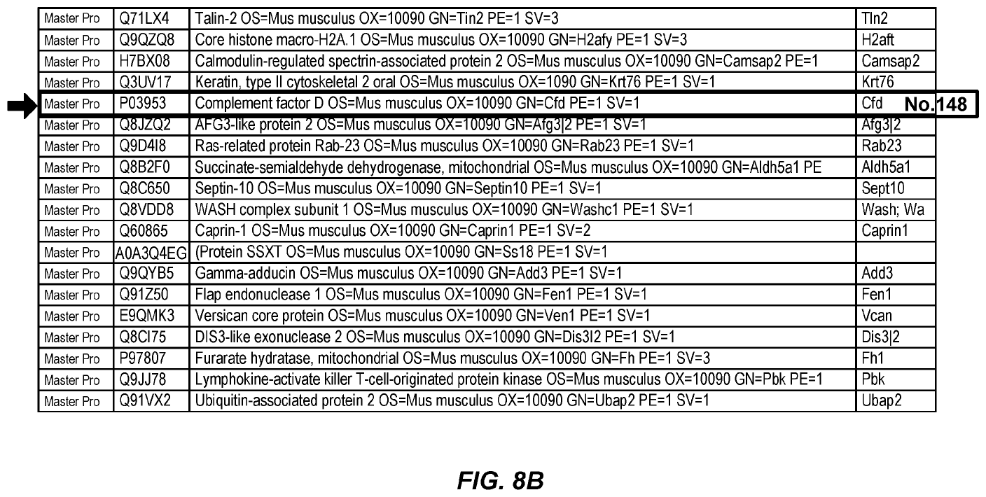

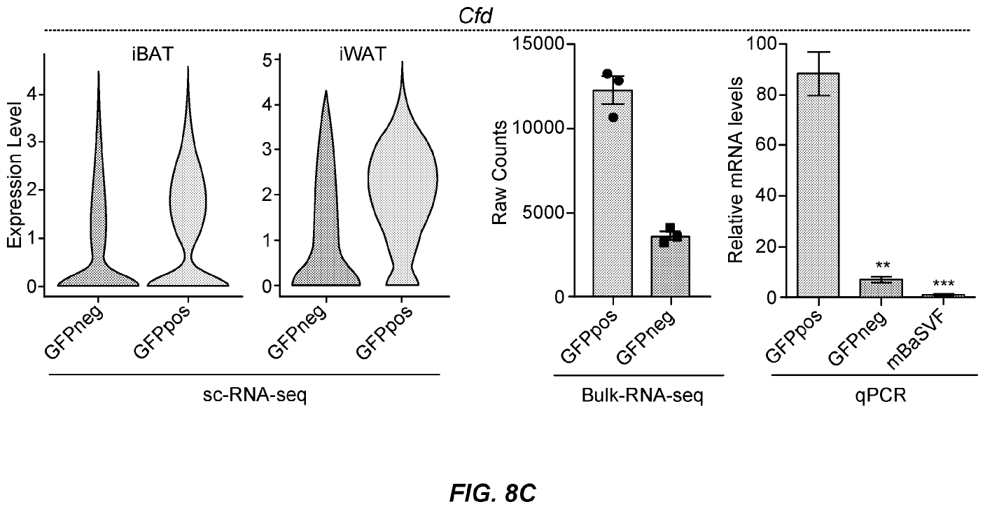

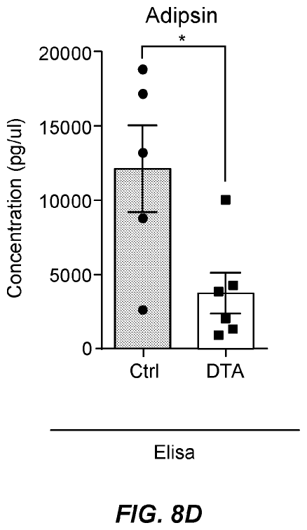

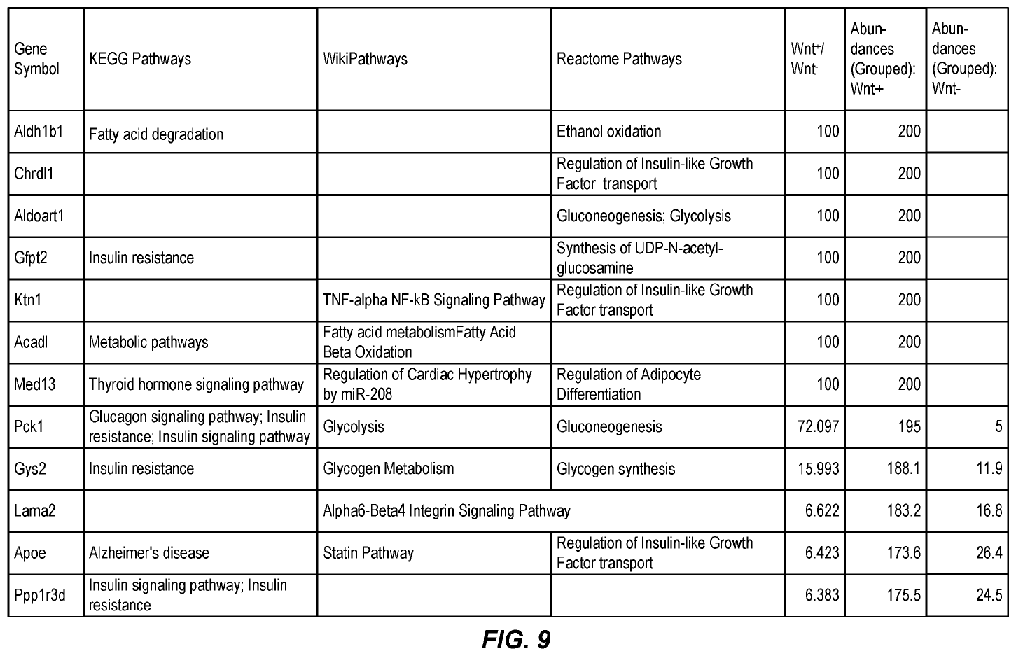

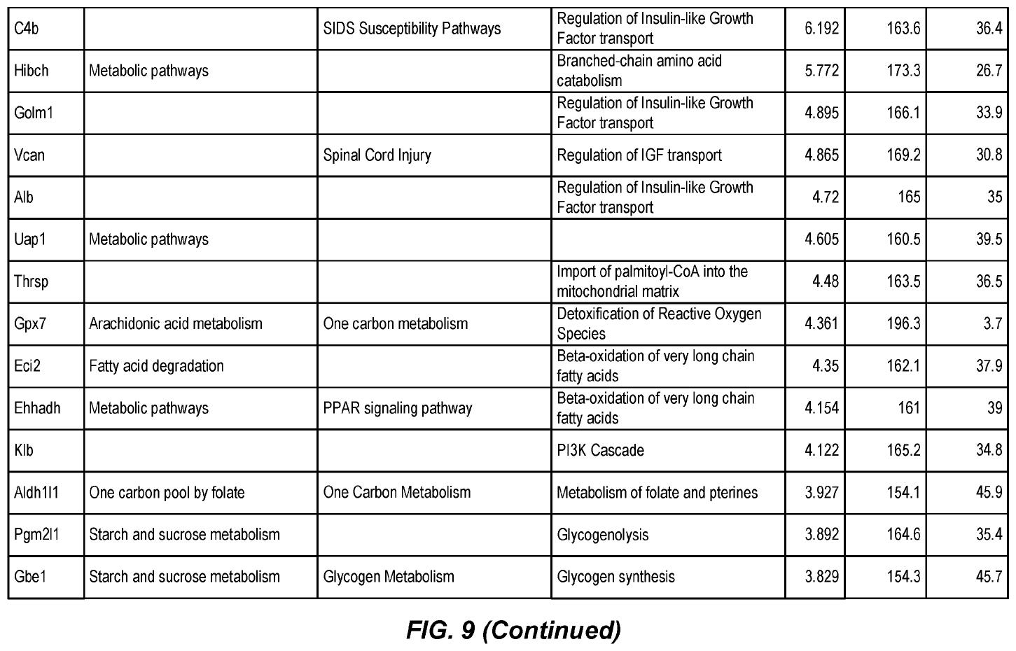

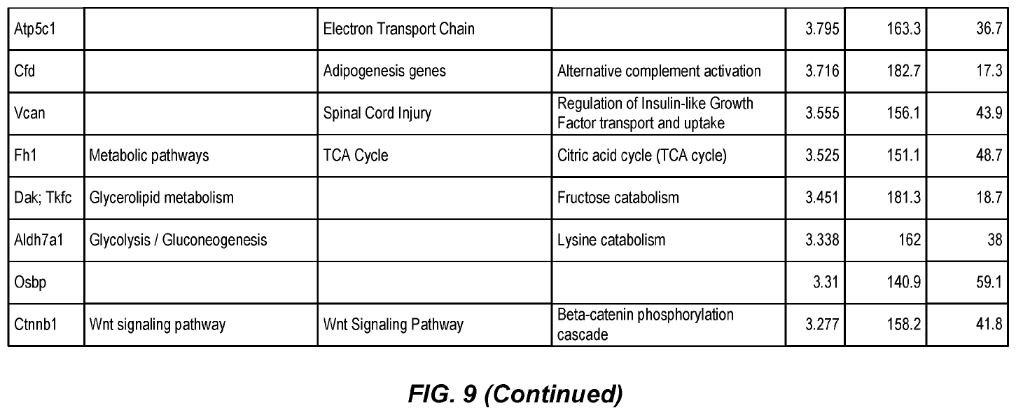

Studies underlying the present disclosure showed that exosomes derived from Wnt⁺ adipocytes have surprisingly higher levels of factors associated with insulin pathways and with glucogenesis and glycolysis than do Wnt⁻ adipocytes. As shown in FIG. 7, mass spectrometry analyses of proteins in exosomes from these two types of adipocytes revealed that Wnt⁺ adipocytes contained proteins associated with insulin pathways and glucose pathways at levels that were multiples of the levels of the same proteins present in exosomes from Wnt⁻ adipocytes. The important adipokine adipsin (shown in the Figure by the name of the gene, Cdf, which encodes it), for example, was present in Wnt⁺ adipocytes at levels almost four times that in which it was present in Wnt⁻ adipocytes.

As noted previously, exosomes are considered to be non-immunogenic. Further, as exosomes derived from Wnt⁺ adipocytes have already been isolated from the Wnt⁺ adipocytes secreting them, the exosomes can be administered to subjects with either T1DM or T2DM and without regard to their insulin status. In some embodiments, the exosomes are administered to a subject with T1DM. In some embodiments, the exosomes are administered to a subject with T2DM. In some embodiments, the exosomes are administered to an obese subject to reduce obesity or to ameliorate hepatic steatosis.

### Compositions and Formulations

Wnt⁺ adipocytes or exosomes secreted by Wnt⁺ adipocytes for delivery by intravenous injection or by or endoscopic methods and dosage formulations and dosage forms for administration of such agents are well known in the art. It is expected that persons of skill are familiar with the considerable literature and guidance that exists with respect to pharmaceutical formulations, as exemplified by texts such as Gennaro, A., REMINGTON'S PHARMACEUTICAL SCIENCES, 18^{th} Ed., (1990), Sheskey, Cook and Cable, eds. HANDBOOK OF PHARMACEUTICAL EXCIPIENTS, 8^{th} Ed. (Pharmaceutical Press, London, 2017), Perrie and Rades, eds., PHARMACEUTICS—DRUG DELIVERY AND TARGETING (Pharmaceutical Press, London, 2012), Chambers-Fox, REMINGTON EDUCATION: PHARMACEUTICS (Pharmaceutical Press, Philadelphia, 2014), and A. Adej are, ed., REMINGTON: THE SCIENCE AND PRACTICE OF PHARMACY, 23^{nd} Ed. (Academic Press, Philadelphia, 2020).

In addition to the Wnt⁺ adipocytes, or isolated exosomes secreted by Wnt⁺ adipocytes, the formulations may include one or more pharmaceutically acceptable excipients, stabilizers, binders, lubricants, fillers, buffers, antioxidants, preservatives, monosaccharides, disaccharides, and other sugars or carbohydrates, including glucose, mannose, sucrose, mannitol, trehalose or sorbitol.

In some embodiments, the invention provides for the use of a composition comprising Wnt⁺ adipocytes for the manufacture of a medicament for reducing blood glucose levels or protecting pancreatic islet cells in a subject in need thereof. In some embodiments, the Wnt⁺ adipocytes are human Wnt⁺ adipocytes. In some embodiments, the invention provides for the use of a composition comprising isolated exosomes secreted by Wnt⁺ adipocytes for the manufacture of a medicament for reducing improving glucose metabolism and regulation or for protecting pancreatic islet cells in a subject in need thereof.

### Routes, Dosing, and Administration

For purposes of the inventive methods, an “effective amount” of an administration of the Wnt⁺ adipocytes or of isolated exosomes secreted from a population of Wnt⁺ adipocytes refers to an amount of the Wnt⁺ adipocytes or of isolated exosomes that, alone or in combination with further doses, produces the desired response. In some embodiments, the exosomes are isolated from a population of adipocytes of which 40%, 50%, 60%, 70% 80%, 90% or 95% or more are Wnt⁺, with each higher percentage stated being preferable to the one before.

With respect to reducing symptoms of T1DM in a subject when administered, an effective amount is an amount that raises the subject's production of insulin or that protects pancreatic islet cells. With respect to protecting an adult from developing T2DM, or slowing an adult's progression to T2DM, an effective amount is an amount that raises the subject's sensitivity to insulin in the blood or that reduces progression to T2DM.

In some embodiments, administration of the Wnt⁺ adipocytes or of the exosomes is by intravenous administration. Intravenous administration is well known in the art, and details and methods of administering compositions by this route of administration are widely taught and available, as exemplified by, e.g., Garhart, et al., GAHART'S 2021 INTRAVENOUS MEDICATIONS: A HANDBOOK FOR NURSES AND HEALTH PROFESSIONALS, 37th Ed. (Elsevier, 2020), and Fulcher and Frazier, INTRODUCTION TO INTRAVENOUS THERAPY FOR HEALTH PROFESSIONALS, (Saunders, 2007).

In some embodiments, administration of the Wnt⁺ adipocytes or of the exosomes is by endoscopic retrograde cholangio-pancreatography, or “ERCP”. As explained on the website of the NIDDK, “ERCP is a procedure that combines upper gastrointestinal (GI) endoscopy and x-rays to treat problems of the bile and pancreatic ducts.” While ERCP is used primarily to diagnose and treat conditions of the bile ducts and main pancreatic duct, in some embodiments of the inventive methods, it is used to introduce the Wnt⁺ adipocytes or of the exosomes directly into the pancreatic duct. This provides a high local concentration of Wnt⁺ adipocytes or of the exosomes without systemic exposure, or with reduced systemic exposure compared to administering the same agents by intravenous infusion. This high local concentration facilitates uptake of the Wnt⁺ adipocytes or of the exosomes, into pancreatic α and 13 cells, while reducing the incidental distribution to, and uptake by, other organs.

Exosomes have been shown to be well tolerated and do not induce toxicity in mice when repeatedly injected. See, e.g., Kalluri and LeBlue, supra. As vesicles of endosomal origin, they do not carry cell surface markers found on the plasma membrane and do not provoke an immune response. Repeated injections of MSC-derived exosomes in a patient in a clinical trial showed the injections to be well tolerated. See, e.g., Kordelas et al., Leukemia, 2014, 28:970-973, doi:10.1038/leu.2014.41

As stated in Torun et al., (Cureus, 2020 Sep. 2; 12(9):e10209. doi: 10.7759/cureus.10209). while ECRP is a “minimally invasive procedure, it is considered relatively safe but still entails a higher risk than other routine endoscopic procedures.” Among the co-morbidities that Torun et al., noted as having some correlations to worse outcomes was diabetes mellitus with complications. Accordingly, practitioners seeking to improve the insulin production in diabetes patients with complications should consider the severity of the diabetes complications in determining whether to use intravenous administration or ECRP to deliver Wnt⁺ adipocytes or of exosomes for such patients. As the practitioners in this art are typically doctors who specialize in diabetes care, and the persons administering ERCP are typically board-certified gastroenterologists with training and experience in performing ERCP, it is expected that making these determinations is within the scope of clinical judgment exercised by practitioners in the care of their patients. It is likewise expected that practitioners can determine the number of Wnt⁺ adipocytes or of exosomes to administer as a matter of clinical practice.

Pancreatitis is the most common complication of ERCP. Several methods have been determined to prevent or reduce the incidence of post-ERCP (“PEP”), including pre-procedure administration of non-steroidal anti-inflammatory drugs (reviewed in Ahmad et al., Cureus, 2020 Aug. 29; 12(8):e10115.doi: 10.7759/cureus.10115, and Puig et al., PLoS One. 2014 Mar. 27; 9(3):e92922.doi: 10.1371/journal.pone.0092922), or a submucosal epinephrine injection in conjunction with rectal indomethacin (see, Torun, et al., Surg Laparosc Endosc Percutan Tech, 2020 Oct. 12, doi: 10.1097/SLE.0000000000000867 (online ahead of print publication). The Ahmad et al. authors concluded that a “combination of rectal NSAIDs and intravenous hydration provides improved prophylaxis against PEP in high-risk patients [compared to] NSAIDs alone” and that “[n]afamostat, sublingual nitrates, and intravenous hydration are potential alternatives in patients with contraindications to NSAIDs.” It is expected that the expert gastroenterologists administering Wnt⁺ adipocytes or of exosomes isolated from a population of adipocytes, 30% or more of which are Wnt⁺ by ERCP will follow the most up-to-date guidelines available at the time of administration to reduce the risk of pancreatitis.

ERCP has also been used to deliver gene therapy agents to the liver through the common bile duct in a large animal model. See, e.g., Kumbhari et al., Gastrointestinal Endoscopy, 2018, 88(4):755-763.e5, doi.org/10.1016/j.gie.2018.06.022. It is expected that this route of delivery can also be used to provide local delivery of Wnt⁺ adipocytes or of exosomes to a subject in need thereof without systemic exposure, or with reduced systemic exposure compared to administering the same agents by intravenous infusion.

### EXAMPLES

### Example 1

This Example describes the materials and methods used in the studies reported in Example 2.

### Animals

Animal studies were performed according to procedures approved by the Institutional Animal Care and Use Committee of Tulane University. C57BL/6J wild-type mice (Stock No. 000664), TCF/Lef:H2B-GFP mice (Stock No. 013752), Rosa26R^{mTmG} mice (Stock No. 007676), and Ins2^{Akita} mice (Stock No. 003548) were purchased from the Jackson Laboratory (Bar Harbor, ME) and maintained on C57/BL6 background. Mice were raised on a standard rodent chow diet and housed at ambient temperature (22° C.) under a 12-hrs light cycles, with free access to food and water. For all experiments, male mice at 8-10 weeks of age were used, unless otherwise stated.

To generate Tcf/Lef-CreERT2 transgenic mice, the coding sequence of the CreERT2 was cloned into pTCF/Lef:H2B-GFP vector by replacing H2B-GFP sequences under the control of the hsp68 minimal promoter. Fabp4-Flex-DTA transgenic construct was generated by inserting the coding sequence of diphtheria toxin A (DTA) into the pBS Fabp4 promoter (5.4 kb) polyA vector (plasmid 11424, Addgene, Watertown, MA), flanked by flip-excision (FLEX) switch. Pronuclear injection and embryo transfer were performed following standard protocols at Tulane Transgenic Animal Facility.

For cold-exposure experiments, mice were singly caged and exposed to cold temperature at 6° C. for 2, 4, or 14 consecutive days. For β3-adrenoceptor agonist treatment, adult male mice were injected intraperitoneally with CL316,243 (CAS No. 138908-40-4, product C5976, Sigma-Aldrich Inc., St. Louis, MO) at 1 mg/kg body weight daily for 4 consecutive days prior to sample collection. Age-matched male littermates were treated intraperitoneally with saline as vehicle controls. For mTOR specific antagonist administration, adult male mice were injected intraperitoneally with Temsirolimus (CAS No. 162635-04-3, product PZ0020, Sigma-Aldrich) at 600 μg/kg (dissolved in 40% ethanol) body weight daily for 5 consecutive days prior to sample harvest. Age-matched male littermates were treated intraperitoneally with 40% ethanol as vehicle controls.

To conduct in vivo lineage-tracing, Tcf/Lef-CreERT2 allele was compounded with Rosa26 R^{mTmG} allele to generate Tcf/Lef-CreERT2;Rosa26 R^{mTmG} mice that received tamoxifen (dissolved in corn oil) administration intraperitoneally at the dose of 100 mg/kg body weight for 5 consecutive days, and samples were harvested at day 6. For cold exposure study, tamoxifen administrated Tcf/Lef-CreERT2;Rosa26 R^{mTmG} mice and littermate controls (Tcf/Lef-CreERT2 mice that received identical tamoxifen treatment) were rested for tamoxifen washout for 2 weeks before they were housed at 6° C. for 4 consecutive days.

To ablate Wnt⁺ adipocytes in vivo, Fabp4-Flex-DTA mice were crossed with Tcf/Lef-CreERT2 mice to generate Tcf/Lef-CreERT2;Fabp4-Flex-DTA mice (“T/L-DTA” mice), which received 2-day tamoxifen administration via intraperitoneal injection at the dose of 150 mg/kg body weight. This dose of 2-day tamoxifen administration was found to significantly ablate Wnt⁺ adipocytes (about 87%) in iWAT of T/L-DTA mice. Forty-eight hours later, T/L-DTA mice and tamoxifen-treated littermate controls (Fabp4-Flex-DTA mice) were housed at 6° C. for 2 days, respectively.

### Isolation of Mouse Adipose Stromal Vascular Fractions (SVFs) and Bone Marrow Stromal Cells (BMSCs)

All cells were isolated from adult TCF/Lef:H2B-GFP male mice, unless otherwise specified. For the isolation of adipose stromal vascular fraction (“SVF”) cells, fat tissues were dissected into ice-cold PBS and minced with scissors in a sterile 5 ml tube containing 4 ml of either iBAT digestion buffer (1× Hanks' Balanced Salt Solution, 3.5% Bovine Serum Albumin (“BSA”), and 2 mg/ml collagenase type II) and incubated at 37° C. for 1 hr under agitation or WAT digestion buffer (1× Dulbecco's phosphate buffered saline (“DPBS”), 1% BSA, 2.5 mg/ml dispase and 4 mg/ml collagenase D) for 40 min. The digestion mixture was passed through a 100 μm cell strainer into a 50 ml tube. Digestion was stopped by adding 15 ml PBS containing 2% fetal bovine serum (“FBS”; catalog no. 10270106, Gibco (Thermo Fisher Scientific, Waltham, MA) and centrifuged at 500 g for 5 min at room temperature. The supernatant was aspirated, and red blood cells were lysed by incubating the SVF pellet with 2 ml RBC lysis buffer (catalog no. 00433357, Invitrogen). The number and viability of cells in the suspension were determined using Trypan Blue stain according to the manufacturer's recommendations.

For isolation of bone marrow stromal cells (“BMSCs”), femurs and tibias were carefully disassociated from muscle, ligaments, and tendons on ice, and then transferred into a sterile 5 ml tube with DPBS containing 2% FBS. Both ends of the bones were cut to expose the interior of the marrow shaft, and bone marrow was flushed out with MesenCult™ Expansion medium (catalog no. 05513, Stemcell Technologies, Inc., Cambridge, MA) using a 6 ml syringe and a #23 gauge needle and collected into a 50 ml tube. Cells were gently resuspended and filtered through a 100 mm filter into a collection tube and cell number and viability were determined as described above.

### Fluorescence-Activated Cell Sorting (FACS)

Freshly isolated single-cell suspensions of SVFs were diluted to 1×10⁷ cells/ml with FACS sorting buffer (DPBS with 2% FBS, 1 mM EDTA) and the following fluorophore-conjugated antibodies were added: CD31/PE, CD45/PE, and Ter119/PE to enrich lineage⁻ (Lin⁻) populations. Cells were incubated with a cocktail of antibodies on ice for 30 min protected from light and subjected to FACS using a Sony SH800 cell sorter (Sony Biotechnology, San Jose, CA). The cells were selected based on their size and complexity (side and back scatter), and then subjected to doublet discrimination to obtain single-cell signal. The Lin⁻ (CD31⁻ CD45⁻ TER119⁻) population was sorted out and plated at the density of 4×10³ cells/cm² for further pro-adipogenic induction.

To sort out Wnt⁺ (GFP⁺) and Wnt⁻ (GFP⁻) adipocytes differentiated from the SVFs for scRNA-seq and scATAC-seq, 7-AAD was used for viable cell gating. Flow cytometric sorting experiments for adipocytes were performed using a FACS-Aria II flow cytometer (BD Biosciences, Becton Dickinson and Co., Franklin Lakes, NJ).

Development of Immortalized Precursors of Wnt⁺ and Wnt⁻ Adipocytes

To establish clonal cell lines of immortalized precursors of Wnt⁺ and Wnt-adipocytes, we generated a lentiviral shuttle vector pUltra-hot-LT that expresses mCherry and Simian Virus 40 Large T antigen (SV40-LT) simultaneously. SV40-LT fragment was amplified from template plasmid pBABE-neo-LargeTcDNA (catalog no. 1780, Addgene) with primers 5′-gactcatctagagataaagttttaaacagagaggaatctttgcagc-3′ (SEQ ID NO:1) and 5′-gcatacggatcctgtttcaggttcagggggagg-3′(SEQ ID NO:2), and the 2145 bp PCR product was subsequently digested with XbaI and BamHI. The vector template pUltra-hot (catalog no. 24130, Addgene) was linearized with XbaI and BamHI as well. Both the digested PCR product and the linearized pUltra-hot vector were gel-purified and ligated with T4 DNA ligase (catalog no. EL0011, Thermo Fisher Scientific) according to manufacturer's instructions. The ligated vector, termed pUltra-hot-LT, is the final shuttle vector.

For lentivirus (LV) production, 60% confluent monolayers of 293-T cells were transfected with LV shuttle vector pUltra-hot-LT and the packaging plasmids psPAX2 (catalog no. 12260, Addgene) and pMD2.G (catalog no. 12259, Addgene) at a molar ratio of 4:3:1. The 293-T cells were cultured in high-glucose Dulbecco's modified Eagle's medium (DMEM; ThermoFisher Scientific, catalog no. 12430112) with 10% FBS (ThermoFisher Scientific, catalog no. 16000069), supplemented with 1% Penicillin-Streptomycin (ThermoFisher Scientific, 15140148) and 1% non-essential amino acids (ThermoFisher Scientific, 11140050). Transfections were performed using a Helix-IN™ transfection kit (catalog no. HX10100, OZ Biosciences Inc., San Diego, CA) following manufacturer's instructions. Forty-eight hrs after transfection, the 293-T culture supernatants were harvested and passed through a 0.45-μm pore-sized, 25-mm diameter polyethersulfone syringe filters (Whatman, 6780-2504). LV particles were concentrated from the filtered supernatants using a Lenti-X concentrator kit (Takara, 631232) following manufacturer's protocol.

SVFs of iBAT from adult TCF/Lef:H2B-GFP male mice were separated and collected as described above. Cells at passage 1 were transduced with pUltra-hot-LT LV particles. Two days after transduction, cells were purified by performing FACS to remove mCherry- and Lin+ cells Immortalized cells were subjected to clonal selection through serial limited dilutions. The expression of GFP in cell colonies under pro-adipogenic induction was used as a readout for the accurate establishment of Wnt+ (>95%) and Wnt− (Wnt+=0%) adipocyte cell lines, respectively.

### In Vitro Pro-Adipogenic Differentiation of Mouse Cells

Immortalized cell lines, isolated adipose SVF cells, MEFs, and BMSCs were seeded into plates at the density of 4×10³ cells/cm² (cell line and SVF) or 1.5×10⁴ cells/cm2 (BMSCs) in MesenCult™ Expansion medium (Stemcell Technologies Inc., Cambridge, MA, catalog no. 05513), and treated with MesenCult™ Adipogenic Differentiation cocktail (Stemcell, catalog no. 05507) with 90% cell confluence. Cells were cultured for various days after pro-adipogenic induction prior to being harvested for following experiments. Adipogenesis was examined by lipid staining. Briefly, differentiated adipocytes were washed twice with PBS, fixed in 4% paraformaldehyde (PFA) for 15 min, and then stained with Oil Red O solution (Sigma-Aldrich, catalog no. 01391) for 20 min at ambient temperature or with 20 nM BODIPY (dipyrrometheneboron difluoride, catalog no. D3835, Thermo Fisher Scientific) in PBS for 15 min at 37° C. Subsequently, cells were washed with PBS before imaging.

For inhibition studies, SVF cells or precursor cell lines, 3 days after pro-adipogenic induction, were treated with each of the following molecules for 4 days: DKK1 (product 5897-DK-010, R&D Systems, Minneapolis, MN) (100 ng/ml), IWP-2 (catalog no. 72122, Stemcell), (5 μM), LF3 (catalog no. 58474, Selleck Chemicals) (10 and 20 μM, or 50 μM on cell lines), and LY294002 (catalog no. S1105, Selleck Chemicals) (2 and 14 μM), along with vehicles (PBS or DMSO).

### SiRNA-Mediated Knockdown Experiment

To knockdown Ctnnb1 in adipocytes, siRNA probes (TriFECTa® DsiRNA Kit) were purchased from Integrated DNA Technologies Inc. (Coralville, IA) and transfected into BMSCs that had been under pro-adipogenic induction for two days. Cells at a density of 5×10⁴ cells/cm² were plated with 10 nM of a given siRNA dissolved in 1.5% Lipofectamine RNAiMAX (Invitrogen, catalog no. 13778150) in Opti-MEM™ I reduced serum medium (Invitrogen, catalog no. 31985062) and pro-adipogenic differentiation medium (STEMCELL, catalog no. 05507). The medium was replaced after 24 hr incubation. Time-lapse imaging was carried out to record the real-time change of GFP signal and cell morphology for the next 2 days. At the end, cells were harvested.

Detection of Activated Wnt/13-Catenin Signaling in Adipocytes Differentiated from Human BMSCs

Primary human bone marrow stromal cells (“hBMSCs”) from a 29-year-old Caucasian male were purchased from Lifeline Cell Technology (Frederick, MD, catalog no. FC-0057 Lot #06333). Lentiviral shuttle vector pUltra-hot-Tcf/Lef:H2B-GFP was generated to serve as a Wnt/β-catenin signaling reporter, with mCherry as a reporter for successful transduction of hBMSCs. The inserted fragment Tcf/Lef:H2B-GFP was amplified from a template plasmid (Addgene, catalog no. 32610) with primers 5′-agagatccagtttggttaattaatattaaccctcactaaagg-3′ (SEQ ID NO:3) and 5′-tggagccgacacgggttaatttacttgtacagctcgtc-3′ (SEQ ID NO:4). The 2335-bp PCR product was subsequently gel-purified. The vector template pUltra-hot (Addgene, catalog no. 24130) was linearized with Pad and gel-purified. The purified PCR products and linearized vectors were assembled using a GenBuilder™ Plus kit (Genscript USA Inc., Piscataway, NJ, catalog no. L00744-10) according to the manufacturer's instructions. The assembled vector was termed pUltra-hot-Tcf/Lef:H2B-GFP as the final shuttle vector. LV production was described as above.

Human BMSCs were plated at a density of 5.0×10⁴ cells/cm² in Stemlife MSC-BM Bone Marrow Medium (Lifeline Cell Technology, catalog no. LL-0026). When cultured cells reached 60-70% confluency, concentrated LV pellets were resuspended and mixed with LentiBlast™ reagent A and B (1:100) (OZ Biosciences, catalog no. LBPX500) in a freshly prepared medium and incubated with cells. Forty-eight hrs after viral transduction, hBMSCs were cultured in either pro-adipogenic induction medium (Lifeline Cell Technology, catalog no. LL-0059) or pro-osteogenic induction cocktail (Lifeline Cell Technology, catalog no. LM-0023) as positive control for Wnt/β-catenin signaling activity for 7 days, respectively. Cells were subsequently subjected to immunofluorescent assays.

### Immunofluorescence

Adipose tissue was fixed in 4% PFA at 4° C. overnight, followed by dehydration through serial ethanol. Samples were processed into paraffin-embedded serial sections at 6 μm. For immunostaining, paraffin-embedded sections were deparaffinized in xylene and subsequently rehydrated. After the incubation of the slides in boiling Tris-EDTA antigen retrieval buffer for 10 min, the tissues were blocked in PBS containing 10% BSA for 60 min, followed by incubation with primary antibodies against GFP (1:500) (Santa Cruz, sc-9996), Perilipin (1:500) (Abcam, ab61682), UCP1 (1:300) (Abcam, catalog no. ab10983), active β-catenin (1:100) (Developmental Studies Hybridoma Bank, Iowa City, IA, product no. PY489-B-catenin), Tcf3 (1:500) (Abcam, ab243152), and Tcf1 (1:100) (CST, catalog no. 2203) at 4° C. overnight. Slides were then incubated with secondary antibodies (1:500) at room temperature for 60 min. After washing, sections were processed with a Vector TrueVIEW™ Autofluorescence Quenching Kit treatment (Vector Laboratories, Inc, Burlingame, CA, SP-8400) for 5 min to remove unspecific fluorescence on sections due to red blood cells and structural elements such as collagen and elastin. Sections were stained with 4′,6-diamidino-2-phenylindole (DAPI) and mounted with mounting medium (Vector, Vibrance™ Antifade). Images of tissue samples were captured using a Nikon confocal Microscope A1 HD25 (Nikon Instruments Inc., Melville, NY) and analyzed using ImageJ software (Version 1.51S). For quantification of Wnt⁺ adipocytes, we randomly chose 12-18 images from each tissue of one mouse. Microscopic pictures (10× objective) were taken, and the total cell number and the number of GFP⁺ adipocytes within each image were counted.

For cryosections, samples were dehydrated in 30% sucrose PBS solution overnight at 4° C., embedded in optimal cutting temperature compound (Tissue-Plus™; Fisher Healthcare), and frozen by liquid nitrogen for solidification. Embedded samples were cryosectioned (Leica, CM1860) at 8 μm and subjected to immunofluorescent staining with primary antibodies against Tcf4 (1:1:00) (Abcam, catalog no. ab76151) and Tcf1 (1:100) as described above.

For immunostaining on cell culture, cells were fixed with 4% PFA for 15 min at ambient temperature and then permeabilized in 0.25% Triton™ X-100 in PBS for 10 min, followed by blocking with 10% BSA in PBS for 30 min. Cells were then incubated with primary antibodies against GFP (1:500), Perilipin (1:500), Runx2 (1:300) (Santa Cruz, sc-390351), mCherry (1:200), Pparγ (1:400) (Abcam, ab45036), Adiponectin (1:100) (Abcam, ab22554), Cyp2e1 (1:200) (LSBio, LS-C313045), and Cidea (1:100) (LSBio, LS-C498624) at 4° C. overnight, respectively. Subsequently, cells were stained with secondary antibodies (1:500) at room temperature for 60 min, followed by counterstaining with DAPI. Images were obtained using a Nikon confocal Microscope A1 HD25 and analyzed using the ImageJ software.

### Western Blotting

Proteins were extracted from iWAT or cultured adipocytes using Minute™ Total Protein Extraction Kit (Invent Biotechnologies, Inc., Plymouth, MN, catalog no. AT-022) according to manufacturer's instructions. 20 μg of proteins were separated by SDS-PAGE (Invitrogen, NW04127BOX) and transferred onto a 0.22 μm Nitrocellulose membrane (LI-COR Biosciences, Lincoln, NE, 926-31092). Membranes were blocked in Tris-buffered saline (TBS) with 0.1% Tween 20 and 5% BSA for 1 hour, followed by overnight incubation with primary antibodies against AKT (1:1,000), p-AKT (1:2,000), GSK-3(3 (1:1,000), p-GSK-3(3 (1:500), 4E-BP1 (1:1,000), p-4E-BP1 (1:1,000), UCP1 (1:1,000), OXPHOS cocktail (1:250), or β-actin (1:10,000) in blocking solution at 4° C. overnight. Samples were then incubated with secondary antibodies conjugated to IRDye 800 or IRDye 680 (LI-COR Biosciences) diluted at 1:5000 for 1 hour Immunoreactive protein was detected by Odyssey Imaging System (LI-COR Biosciences).

### RNA Preparation and Quantitative RT-PCR

Total RNA was extracted from tissue or cells according to protocol for the RNeasy Lipid Tissue Mini Kit (Qiagen, Germantown, MD, catalog no. 74804) or RNeasy Mini Kit (Qiagen, catalog no. 27106) accordingly. Complementary DNA was synthesized using RevertAid First Strand cDNA Synthesis kit (Thermo Scientific™, catalog no. K1622) according to the protocol provided. Quantitative PCR was performed using a C1000 Touch™ thermal cycler (BioRad, Hercules, CA, CFX96 ™ Real-Time System). Values of each gene were normalized to reference genes, 36B4 or Adipoq, using the comparative Ct method.

ScRNA-Seq and scATAC-Seq

SVF cells isolated from iBAT (3 males and 4 females) and iWAT (10 males and 10 females) of adult L/T-GFP mice were cultured and induced to differentiate into adipocytes as described above. Following separation by FACS, approximately 3,000 iBAT-derived GFP⁺, iBAT-derived GFP⁻, iWAT-derived GFP⁺ cells each, and 4,000 iWAT-derived GFP⁻ cells were loaded using a Chromium Single Cell 3′ v2 Reagent Kit (10× Genomics, Inc., Pleasanton, CA), and libraries were run on the Illumina NextSeq 550 Sequencing System (Illumina Inc., San Diego, CA) as 150-bp paired-end reads. Sequencing data were processed on CellRanger v.3.1.0 (10× Genomics) pipeline with default parameters, including demultiplexing, conversion to FASTQs using bcl2fastq2 v.2.27.1 software, alignment to the mm10 mouse reference genome, filtering, and unique molecular identified (UMI) counting. Gene expression count matrixes were obtained with 2,329 iBAT GFP cells (32,539 mean reads and 1,313 median genes per cell), 2,666 iBAT GFP⁻ cells (29,534 mean reads and 2,076 median genes per cell), 2,318 iWAT GFP cells (35,693 mean reads and 2,282 median genes per cell), and 3,137 iWAT GFP⁻ cells (24,360 mean reads and 2,565 median genes per cell). Further scRNA-seq data analysis was performed using a Seurat package v.3.2.2 (Satija lab, New York Genome Center, New York, NY). Low-quality cells with fewer than 500 detected genes, doublets with more than 4000 transcripts, and cells with mitochondrial fraction rate higher than 40% were excluded from the analysis. Normalized and scaled data were clustered using the top 20 significant principal components of highly variable genes with the parameter “resolution=0.7”. Uniform Manifold Approximation and Projection (UMAP) dimensionality reduction was performed to visualize the resulting clusters. To filter out non-adipocytes, preadipocytes, and endothelial cells, cell clusters identified by high expression levels of precursor marker Pdgfra55 and endothelial marker Cdh556 were removed, and Adipoq-expressing cells were considered as adipocytes57 that were reperformed to assign cluster. The final reported datasets consist of 1,710 Wnt⁺ and 827 Wnt⁻ adipocytes for iBAT, 984 Wnt⁺ and 361 Wnt⁻ adipocytes for iWAT, respectively. Top marker genes of each cluster and library were determined by the Seurat FindAllMarkers function with the parameters “only.pos=TRUE, min.pct=0.2, logfc.threshold=0.25”. Heat maps of the top 30 enriched genes from each library was generated using the Seurat DoHeatmap function. Hallmark gene sets, GO Biological Processes ontology, KEGG pathway, and Wikipathway analyses were performed using the Molecular Signatures Database (MSigDB) maintained by the Gene Set Enrichment Analysis project (https://www.gsea-msigdb.org/gsea/msigdb) to estimate the functional enrichment and biological pathway of input Wnt⁺ adipocytes based on the differential gene expression (FDR<0.05).

For the scATAC-seq experiment, SVF cells from adult L/T-GFP mice (11 males for iBAT; 7 males and 2 females for iWAT) were induced and adipocytes were acquired as set forth in t the workflow for scRNA-seq. To isolate nuclei, cells were lysed for 4 min on ice according to a 10× Genomics protocol (CG000169 Rev D). ScATAC-seq libraries were prepared using the 10× Genomics platform with a Chromium Single Cell ATAC Library & Gel Bead Kit as recommended by the manufacturer and sequenced on an Illumina NextSeq 550 Sequencing System as 150-bp paired-end reads. Peak matrixes and metadata were generated by Cell Ranger ATAC v.1.1.0 pipeline with default parameters and aligned to the mm10 mouse reference genome. Overall, scATAC-seq datasets containing 3,284 iBAT GFP cells (17,706 median fragments per cell), 4,650 iBAT GFP⁻ cells (9,994 median fragments per cell), 766 iWAT GFP cells (35,731 median fragments per cell), and 1,034 iWAT GFP⁻ cells (42,083 median fragments per cell) were obtained. Further data analysis was performed using the Signac v.1.0.0 package (Signac is an extension of Seurat software for the analysis, interpretation, and exploration of single-cell chromatin datasets, by the Satija lab of the NY Genome Center). In brief, outlier cells with <2,000 or >40,000 peaks (iWAT GFP⁺>60,000 peaks, iWAT GFP⁻>90,000 peaks), <20% reads in peaks, >0.025 blacklist ratio, >4 nucleosome signal or TSS-enrichment <2, were considered as low-quality cells or doublets and were removed from downstream analyses. Data of each experiment based on the 95% most common feature were then normalized and scaled through the FindTopFeatures and RunSVD functions. UMAP and k-nearest neighbor (KNN) were applied to perform non-linear dimension reduction using latent semantic indexing (LSI) and clustering analysis with the parameter of “resolution=0.7”. To exclude potential non-adipocytes, gene activity matrix for each experiment was generated by summarizing the accessibility in promoter (TSS and up to 2 kb upstream) and cells with high activity of the adipocyte specific marker Adipoq (the gene encoding adiponectin) were retained for further analysis. We merged the datasets of Wnt⁺ and Wnt⁻ adipocytes derived from iBAT and iWAT, respectively, and applied UMAP for the data visualization. Differential chromatin accessibilities between Wnt⁺ and Wnt⁻ adipocytes were visualized by the CoveragePlot function, and motif activities were computed through Chromvar within Signac.

### Mitochondrial Membrane Potential

Mitochondrial membrane potentials of Wnt⁺ adipocytes were measured through MitoTracker™ Deep Red (far red-fluorescent dye) staining. Briefly, SVF cells derived from iBAT were isolated and collected with the method described above. After 7 days of pro-adipogenic induction, cells were incubated with 100 nM MitoTracker™ Deep Red FM (Invitrogen, catalog no. M22426) for 30 min at 37° C. After washing twice with PBS, GFP (abs/em˜488/509 nm) fluorescence of differentiated adipocytes and MitoTracker™ Deep Red (abs/em˜644/665 nm) of active mitochondria were monitored using a Nikon confocal Microscope A1 HD25 and analyzed using ImageJ software.

### Respiration Measurements

Cellular oxygen consumption rate (“OCR”) was measured using a Seahorse XFe24 analyzer (Agilent Technologies, Inc., Santa Clara, CA). GFPpos-1, GFPpos-2, GFPneg-1, and mBaSVF cells were seeded in an XF24 cell culture microplate (Agilent, product no. 102342-100) at a density of 20,000 cells per well. After 5 days of pro-adipogenic induction, 500 μl XF assay medium containing 1 mM pyruvate, 2 mM glutamine, and 10 mM glucose was added to each well. Cells were subjected to a mitochondrial stress test by adding oligomycin (5 μM), FCCP (1.25 μM), and rotenone and antimycin A (5 μM) according to the manufacturer's instructions (Agilent, product no. 103015-100).

### Core Body Temperature Measurement

Core body temperatures of T/L-DTA mice and control littermates were obtained at room temperature (“RT”), 6-, 12-, 24-, 36-, 48-, 60-hour in cold exposure using a rectal thermometer (Kent Scientific Corp., Torrington, CT, catalog no. WD-20250-91) and rectal probe (Kent Scientific, catalog no. RET-3). Before recording temperatures, measuring instruments were calibrated using ice-water bath each time. The probe was gently inserted at least 2 cm into the rectum of the mice being measured.

### Intraperitoneal Glucose Tolerance Test

Mice were fasted for 6 hours starting from 9 am to 3 μm on the testing day. Glucose was administered intraperitoneally (2 g/kg body weight) and blood glucose levels were determined from tail vein blood samples using an ACCU-CHEK® active glucometer (Roche Diagnostics, Indianapolis, IN) at several time points post glucose injection.

### Cell Implantation

Immortalized GFPpos and mBaSVF cells were differentiated under pro-adipogenic conditions, respectively, as described above. After 2-day induction, 1.3×10⁶ of each group of committed cells were gently re-suspended and embedded in 110 μl Matrigel® (Corning Life Sciences, Tewksbury, MA, catalog no. 356231). The complex was subsequently injected subcutaneously into the left abdomen of C57BL/6J wild-type mice at 8 weeks of age. To trace the fate and the locations of implanted cells, blue agarose beads (150-300 μm in diameter; Bio-Rad, catalog no. 153-7301) were included as an indicator in the implanted complexes. Mice were then kept under regular chow diet at ambient temperature, and ectopically formed fat pads were identified and confirmed by gross and histological examinations 2-week after injection. Accordingly, cell-implanted mice were subject to GTT at 2-week time point after cell implantation.

### Quantification and Statistical Analysis

Statistical analyses were performed using GraphPad Prism 9.0 (GraphPad Software, San Diego, CA), and Excel® software (Microsoft Corp., Redmond, WA). All data were represented as mean±s.e.m, except where noted. A two-sample unpaired Student's t-test was used for two-group comparisons. One-way ANOVA followed by Tukey's test was used for multiple group comparisons, two-way repeated-measures ANOVA followed by Bonferroni's test was used for Seahorse measurement and GTT results from multiple groups. P values below 0.05 were considered significant throughout the study and is presented as *P<0.05, **P<0.01, or ***P<0.001.

### Example 2

This Example discusses the results of studies conducted using the materials and methods set forth in Example 1.

### A Unique Fraction of Murine Adipocytes Displays Wnt/13-Catenin Signaling Activity

Active Wnt/β-catenin signaling was surveyed in developing and mature adipocytes, taking advantage of the Wnt/β-catenin signaling specific reporter mouse line TCF/Lef:H2B-GFP (hereafter “T/L-GFP mouse”) that allows real-time monitoring of Wnt/β-catenin activity at single-cell resolution. Surprisingly, a subset of Perilipin⁺ adipocytes that exhibited Wnt/β-catenin signaling activity was observed interspersed within various fat depots, including interscapular brown adipose tissue (iBAT), inguinal white adipose tissue (iWAT), epididymal white adipose tissue (eWAT), and bone marrow (BM), in adult mice. Since adipose tissues possess multiple types of non-adipocytes, and the nuclei are often squeezed at the edge of mature adipocytes, it is difficult to determine the exact hosts of the GFP⁺ nuclei in adipose tissues. To confirm that these Wnt/β-catenin positive cells were indeed adipocytes, a Tcf/Lef-CreERT2 transgenic allele was created in which the CreERT2 cassette was placed under the control of the same regulatory elements as that used in the T/L-GFP allele. This Tcf/Lef-CreERT2 allele, upon compounding with Rosa26R^{mTmG} reporter allele followed by tamoxifen administration, indelibly labeled the membrane of a fraction of adipocytes, as seen in freshly isolated cells and in sectioned iWAT and iBAT. Moreover, addition of the T/L-GFP allele to Tcf/Lef-CreERT2;Rosa26R^{mTmG} mice produced adipocytes that were tagged by both membrane-bound and nucleus-localized GFP. Therefore, a population of adipocytes was identified that displays active Wnt/β-catenin cascade, and the cells of this population are sometimes referred to herein as “Wnt⁺ adipocytes.”

To chart a census of Wnt⁺ adipocytes within distinct mouse fat depots, Wnt⁺ fat cells were quantified in T/L-GFP mice. It was observed that the adipocytes expressing nuclear green fluorescent protein (“GFP”) account for various percentages of total fat cells in different fat depots, with the highest level (17.02%±3.06%) seen in iBAT the lowest one (1.28%±0.56%) in eWAT, and a relatively abundant percentage (6.86%±0.98%) in iWAT, a representative beiging site of adult male mice. Wnt⁺ adipocytes could be detected as early as embryonic day 17.5 (E17.5) and the proportions varied in different fat depots over the course of post-natal stages. Interestingly, sex and age appeared to have an impact on the number of Wnt⁺ adipocytes, as the percentage of Wnt⁺ adipocytes in female fat depots was around half as compared to their male counterparts and was dramatically reduced in aged male mice.

Characterization of Wnt⁺ Adipocytes Demonstrates their Distinct Lineage Origin and Cellular Intrinsic Wnt/13-Catenin Signaling Activity

To characterize Wnt⁺ adipocytes, studies were conducted to determine if the resident SVF cells within adipose tissues constitute the source of Wnt⁺ adipocytes. Using standard white fat pro-adipogenic induction protocol, Wnt⁺ adipocytes were readily induced in vitro from stromal vascular fractions (“SVFs”) derived from iBAT, iWAT, eWAT, as well as BM stroma, as confirmed by staining of the general adipocyte marker adiponectin, and also surprisingly by the co-expression of Pparγ that is believed to be suppressed by the canonical Wnt signaling. The induced Wnt⁺ adipocytes exhibited morphology similar to Wnt⁻ (non-GFP labeled) ones and also made up a very similar percentage of total induced fat cells as they are present in their corresponding fat depot, suggesting that the Wnt⁺ adipocytes that were observed in vivo are derived from the adipogenic precursors among SVFs. Given the fact that Wnt⁺ adipocytes are present in embryos, it was asked if embryonic mesenchymal stem cells can differentiate to Wnt⁺ adipocytes as well. Notably, E13.5 mouse embryonic fibroblasts (MEFs) could be induced to differentiate into Wnt⁺ adipocytes in vitro, indicating an early developmental origin of Wnt⁺ adipocytes.

Determining if Human Wnt⁺ Adipocytes could be Induced from Stromal Cells

To determine if Wnt⁺ adipocytes found in mice in the studies above would also exist in humans, human primary bone marrow stromal cells (BMSCs) were transfected with an mCherry-expressing lentiviral vector carrying a TCF/Lef:H2B-GFP reporter cassette, and the cells were then cultured with pro-differentiation medium. As a positive control, pre-osteocytes/osteocytes differentiated from infected BMSCs under pro-osteogenic conditions exhibited overlapped mCherry expression and nuclear Wnt/β-catenin signaling activity, validating the effectiveness of the transduction system. Importantly, under the pro-adipogenic medium, GFP-tagged adipocytes from the infected BMSCs were observed, indicating the differentiation of Wnt⁺ adipocytes from human stromal cells. Together, these results suggest that Wnt⁺ fat cells appear to constitute a widespread adipocyte population that originates from embryonic stage and exist in both mice and humans.

### Monitoring Induced Wnt⁺ Adipocytes In Vitro

To address how Wnt⁺ adipocytes develop during adipogenesis, real-time monitoring on behaviors of induced Wnt⁺ adipocytes was conducted in vitro. No Wnt/β-catenin signaling activity was seen in SVFs in culture prior to pro-adipogenic induction. Nuclear GFP expression was initially detected in differentiated adipocytes (defined by the presence of lipid droplets) after 2-day adipogenic induction and the GFP signal was sustained once it was activated, indicating that Wnt/β-catenin signaling does not transiently appear but persists in Wnt⁺ adipocytes. Interestingly, after 7-day differentiation, colonized Wnt⁺ and Wnt-adipocytes, respectively, induced from primary BMSCs of T/L-GFP mice were observed, suggesting distinct cell lineages of these two different adipocyte populations. This conclusion is supported by the observation that induced Wnt⁺ and Wnt⁻ adipocytes, after separation by fluorescence-activated cell sorting (FACS) and subsequent 4-day in culture, did not convert mutually. The consistent Wnt/β-catenin signaling within Wnt⁺ adipocytes thus represents an intrinsic cascade activity.

To ensure that the GFP expression in T/L-GFP adipocytes both in vivo and in vitro represents authentic Wnt/β-catenin signaling activity, the presence of active β-catenin and Tcf/Lef1 in the nuclei of Wnt⁺ adipocytes within adipose tissues was confirmed. It was further found that Tcf712 expression was largely overlapped (˜91%) with Wnt⁺ adipocytes, whereas other Tcf proteins (Tcf1, Tcf3, and Lef1) were barely seen in Wnt⁺ adipocytes (data not shown). Next, knockdown experiments were conducted using short interfering RNA (siRNA) targeting Ctnnb1, followed by real-time monitoring. Ctnnb1 knockdown in differentiated Wnt⁺ adipocytes virtually quenched the GFP signals, followed by cell shrinkage. Moreover, while DKK1 and 1WP-2, both canonical Wnt receptor antagonists, failed to, LF3, a molecule that disrupts the interaction between β-catenin and Tcf712 (Fang et al., Cancer Res, 2016, 76:891-901), did inhibit Wnt/β-catenin signaling activity in precursors under pro-adipogenic conditions This observation indicates that the activation of the cellular intrinsic Wnt/β-catenin signaling in Wnt⁺ adipocytes is ligand- and receptor-independent.

Remarkably, pharmacological inhibition of Wnt/β-catenin signaling by LF3 also resulted in an overall impaired/delayed adipogenic maturation of SVF-derived adipocytes in a dose-dependent manner, suggesting a potentially functional importance of the intracellular Wnt/β-catenin signaling in adipogenesis.

To further define the specific roles of Wnt/β-catenin signaling in Wnt⁺ and Wnt⁻ adipocytes, SVF cells (mBaSVF) derived from iBAT of T/L-GFP mouse were immortalized. By serial limited dilutions, we isolated two Wnt⁺ (GFPpos-1 and GFPpos-2) and two Wnt⁻ (GFPneg-1 and GFPneg-2) adipocyte precursor cell lines from mBaSVF cells, respectively, were isolated. Of note, LF3 treatment of induced Wnt⁺ adipocytes from both GFPpos-1 and GFPpos-2 cell lines quenched GFP expression in the first day of culture, followed by massive cell death in the second day. By contrast, LF3 administration to induced Wnt⁻ adipocytes from precursor cell lines did not affect cell viability but slowed down the maturation of Wnt⁻ adipocytes similar to that seen in SVF induced adipocytes. However, such LF3-treated Wnt⁻ adipocytes, once returned to normal pro-adipogenic medium, resumed full adipogenic capacity compared to controls, indicating that LF3 treatment does not impair but delays the maturation of Wnt⁻ adipocytes. As controls, the same dose of LF3 showed no impacts on uninduced Wnt⁺ and Wnt⁻ precursor cell lines.

Wnt⁺ Adipocytes are Different from Conventional Ones, with Distinct Molecular and Genomic Characteristics

To further distinguish Wnt⁺ adipocytes from Wnt⁻ fat cells and explore the global diversity at molecular and genomic levels, single-cell RNA sequencing analysis (scRNA-seq) and single-cell assay for transposase-accessible chromatin sequencing (scATAC-seq) were performed on FACS separated Wnt⁺ and Wnt⁻ adipocytes induced from iWAT- and iBAT-derived SVF cells, respectively. After filtering for quality and excluding non-adipocytes (negative for adiponectin expression), bioinformatic analyses classified the input Wnt⁺ and Wnt⁻ adipocytes into distinct clusters, indicating two different types of adipocytes. Notably, although the expression of Ucp1 was undetectable in the single-cell transcriptomic data, violin plots showed that several thermogenic-related genes, such as Cox8b, Elovl3, were present at significantly higher levels in Wnt⁺ adipocytes that were subject to the white fat differentiation condition without browning stimuli, indicative of potentially thermogenic character. Moreover, Cyp2e1, a molecule identified as a marker in thermogenic regulation, and Cidea were exclusively expressed in Wnt⁺ adipocytes induced from iWAT- and iBAT-derived SVFs, as confirmed by immunofluorescent staining. These facts link Wnt⁺ adipocytes to thermogenic function in adipose tissues. Enrichment pathway analyses of differentially expressed genes (“DEGs”) also suggests the primary functions of this population of adipocytes in the regulation of adipogenesis, fatty acid metabolism, and mTORC1 signaling. These results demonstrated that this novel population of Wnt/β-catenin signaling-driven adipocytes is distinct from the classic adipocytes at the molecular and genomic levels.

Insulin/AKT/mTORC1 Signaling is Required for Activation of Intracellular Wnt/β-Catenin Cascade within Wnt⁺ Adipocytes

PI3K/AKT/mTOR signaling modulated by insulin is manifested for promoting cytoplasmic β-catenin accumulation through GSK-3β phosphorylation (Hermida et al., Adv Biol Regul, 2017, 65:5-15; Schakman et al., Endocrinology, 2008, 149:3900-3908). This notion of signaling crosstalk was of interest because insulin signaling plays pivotal roles in mediating adipogenic differentiation and functionality and the enrichment pathway analyses in the studies herein also implicate a link to mTORC1 signaling. Accordingly, AKT signaling activities in induced Wnt⁺ and Wnt⁻ adipocytes were assessed Immunoblot assays showed that GFPpos-derived Wnt⁺ adipocytes exhibited markedly higher levels of AKT phosphorylation than that from GFPneg-derived Wnt⁻ adipocytes and from mBaSVF-induced adipocytes as unbiased fat cell controls. Phosphorylated GSK-3β and 4E-BP1 (Eukaryotic translation initiation factor 4E-binding protein 1), a known substrate of mTOR signaling pathway, were also found preferentially higher in Wnt⁺ adipocytes as compared to controls. These results demonstrate enhanced AKT/mTORC1 cascade activity and insulin sensitivity in Wnt⁺ fat cells.

To test if AKT/mTOR signaling is responsible for intracellular Wnt/β-catenin signaling activation in Wnt⁺ adipocytes, iBAT-derived SVF cells were treated with LY294002, a selective PI3K signaling antagonist, under pro-adipogenic conditions. Again, dramatically reduced number of Wnt⁺ adipocytes and blunted adipogenesis were observed, in a dose-dependent manner Consistently, LY294002 administration to GFPpos-induced Wnt⁺ adipocytes eliminated GFP expression and caused subsequent substantial cell death, but did not impact the survival of GFPneg-derived adipocytes. To determine a causal role of mTOR signaling in the intracellular activation of Wnt/β-catenin signaling in adipocytes in vivo, T/L-GFP adult mice were subjected to treatment with an mTOR-specific antagonist, temsirolimus, which dramatically reduced Wnt⁺ adipocyte numbers in both iWAT and iBAT compared to vehicle-injected controls. These results demonstrate the dependence of the Wnt/β-catenin signaling activity in adipocytes on AKT/mTOR signaling, which is required for cell survival.

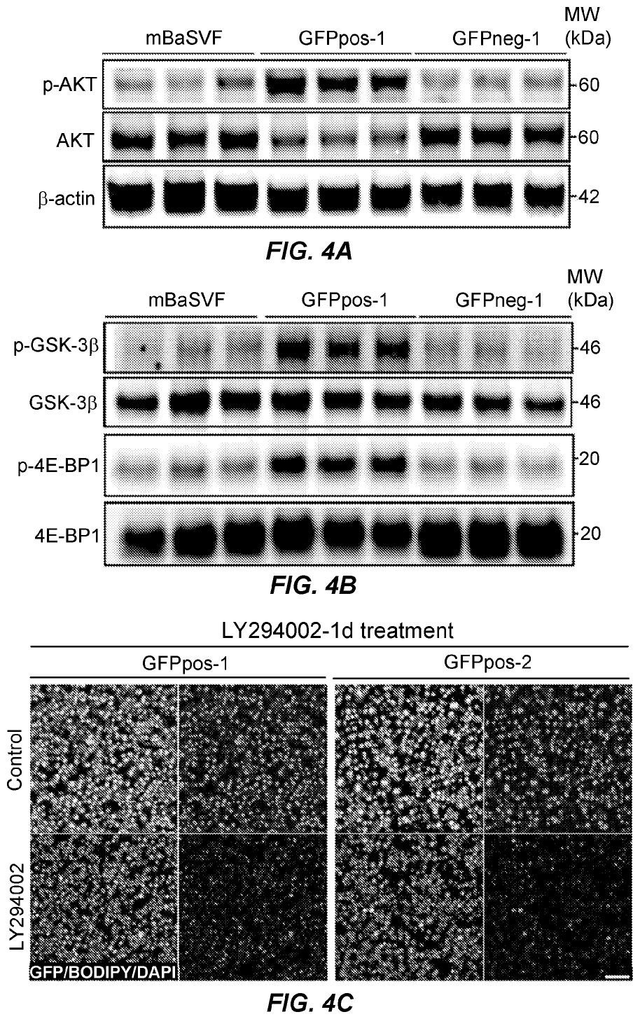

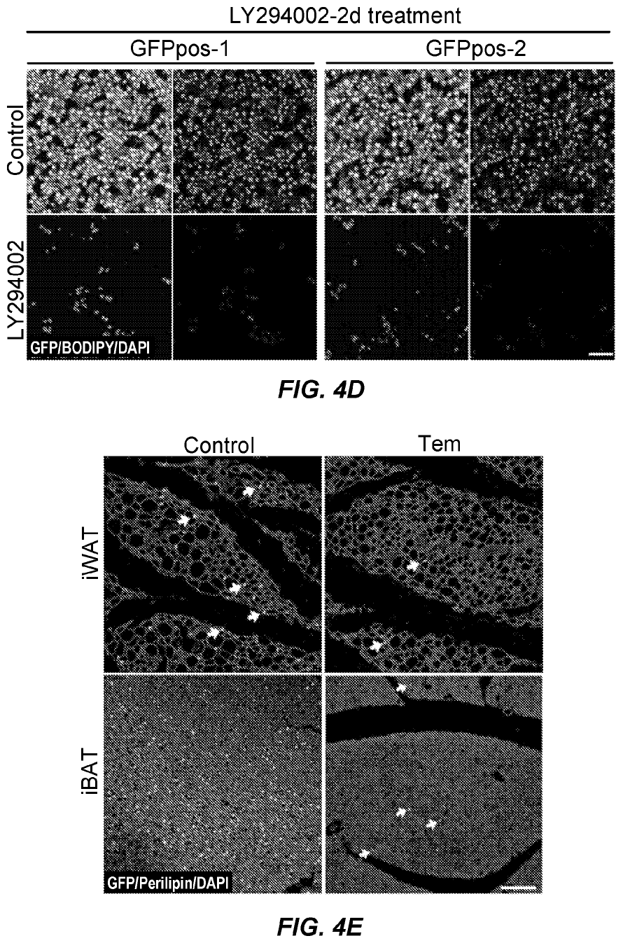

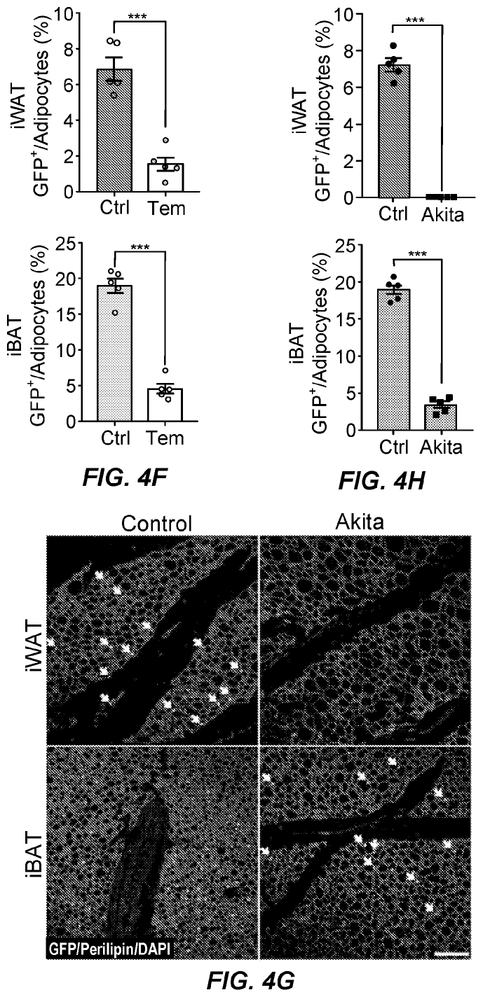

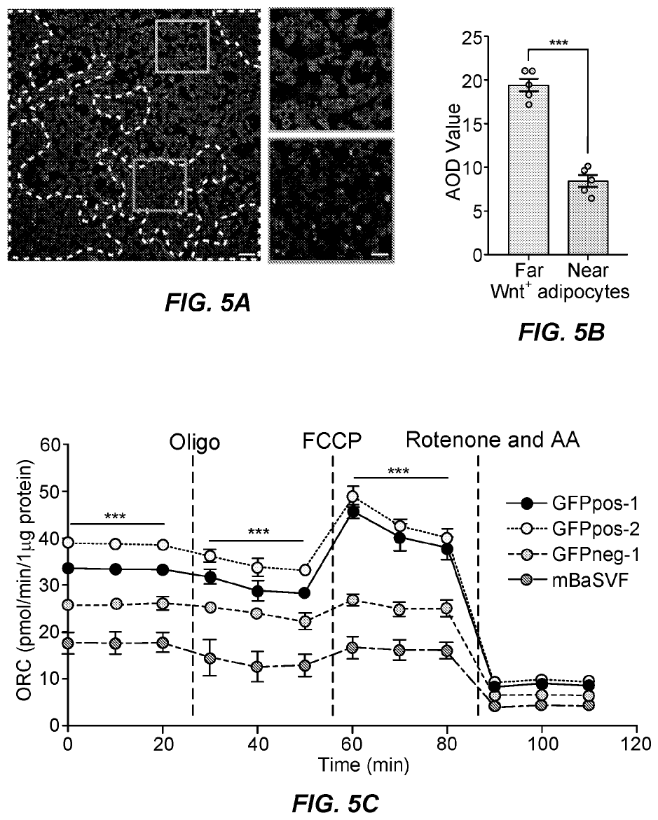

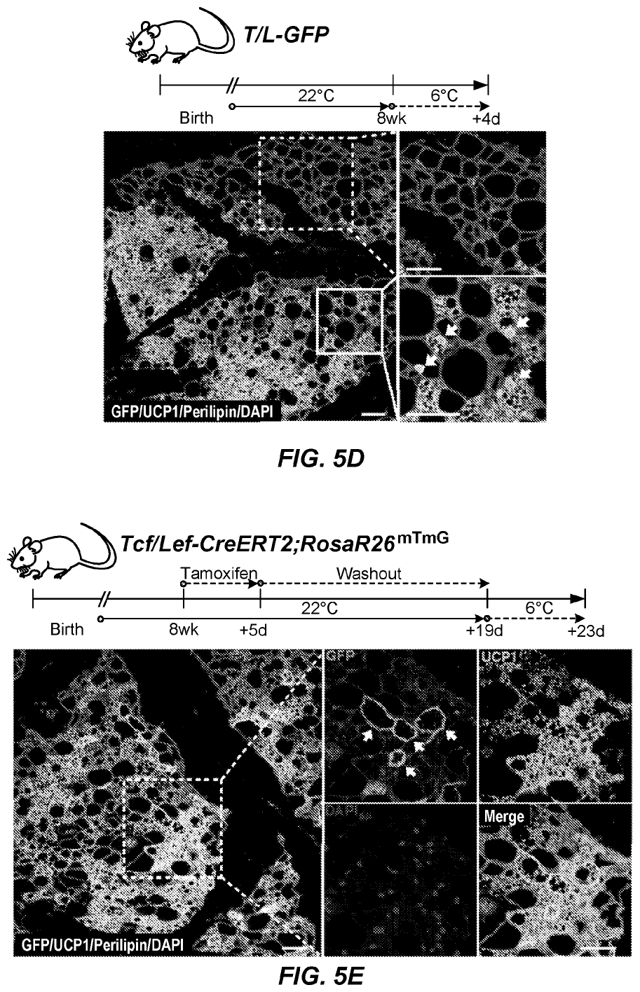

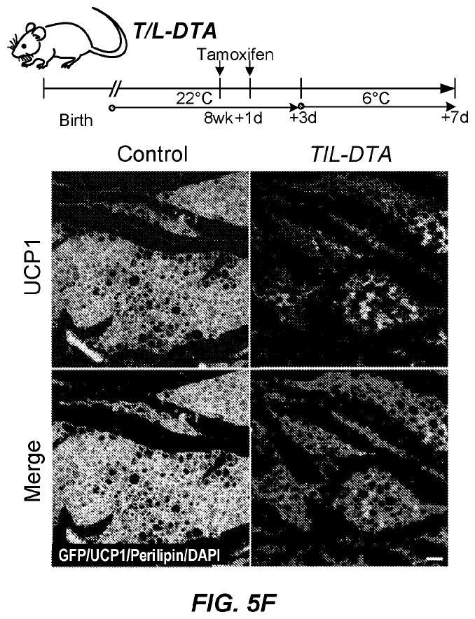

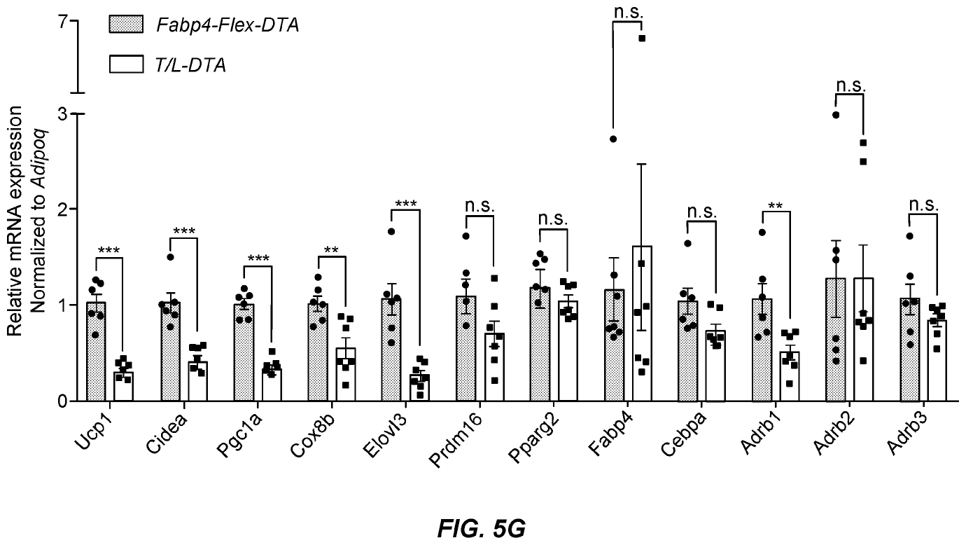

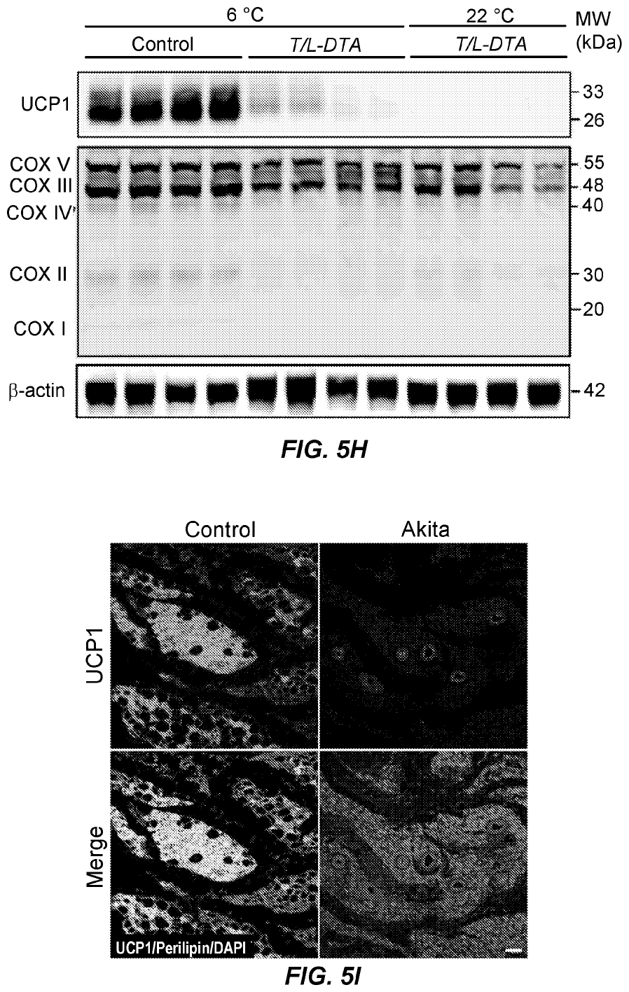

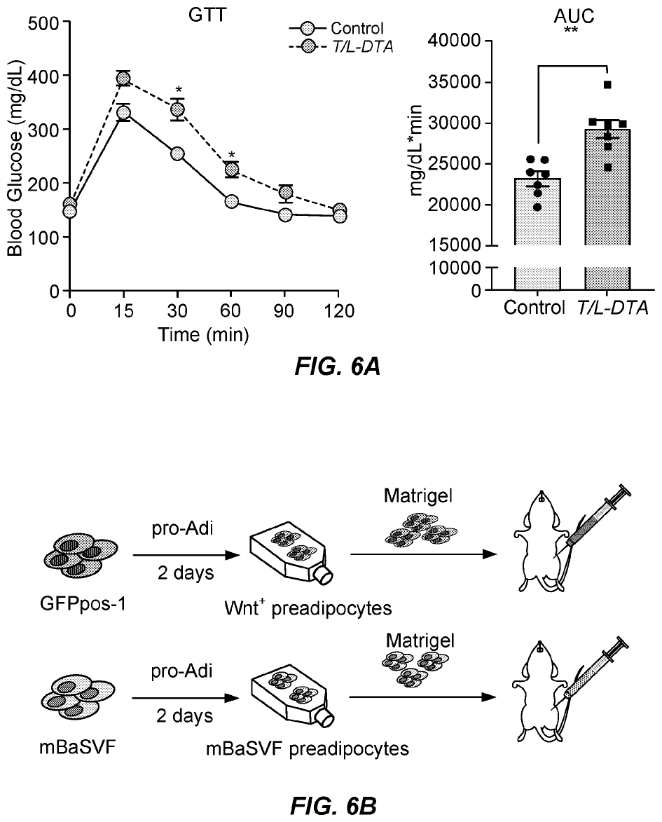

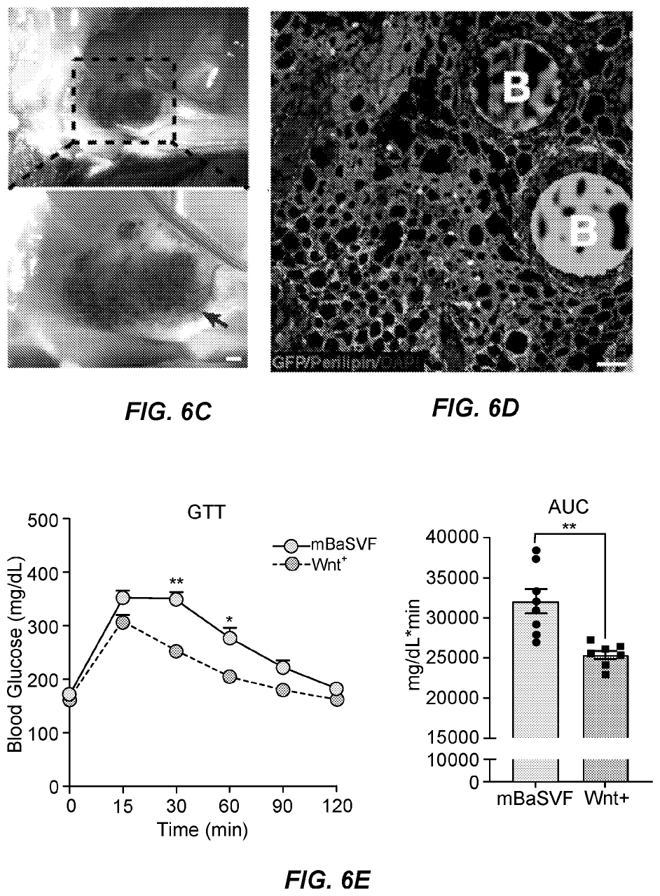

To further validate the requirement of insulin signaling in Wnt⁺ adipocyte development in vivo, Ins2Akita mice (so-called “Akita mice”) were employed. Insulin secretion in Akita mice is profoundly impaired due to a genetic defect in the insulin 2 gene (Yoshioka et al., Diabetes, 1997, 46:887-894), by crossing them to T/L-GFP mice. As expected, Wnt⁺ adipocytes were completely absent in iWAT and substantially reduced in number in iBAT (3.34%±0.55%) of 8-week-old male Akita mice (FIGS. 4G and 4H), demonstrating an indispensable effect of insulin-induced AKT/mTOR signaling on Wnt⁺ adipocyte differentiation.

### Wnt⁺ Adipocytes are Required for Initiating Adaptive Thermogenesis in Both Cell Autonomous and Non-Cell Autonomous Manners

The potentially highly metabolic and thermogenic characteristics of Wnt⁺ adipocytes prompted the investigation of the adaptive thermogenic role of Wnt⁺ adipocytes. The mitochondrial activities in SVF-derived adipocytes were examined in vitro and it was found that Wnt⁺ adipocytes, along with those closely adjacent Wnt⁻ ones, exhibited pronounced lower levels of mitochondrial membrane potential, indicative of higher uncoupling rate, as compared to those Wnt⁻ fat cells located relatively away from Wnt⁺ adipocytes. In addition, significantly higher levels of oxygen consumption rate (OCR) were detected in GFPpos-induced Wnt⁺ adipocytes as compared to those GFPneg-derived Wnt⁻ adipocytes and mBaSVF-induced fat cells, further demonstrating a higher mitochondrial respiration capacity of Wnt⁺ adipocytes.

To explore the possible involvement of Wnt⁺ adipocytes in adaptive thermogenesis in vivo, adult T/L-GFP male mice were subjected to 6° C. temperature for 2 days to initiate beiging. After cold exposure, the presence of a subset of UCP1-expressing beige adipocytes was observed that were also GFP-positive, demonstrating that a portion of beige fat cells arises from Wnt⁺ adipocyte lineage. Similar results were seen in mice treated with the β3-adrenergic receptor agonist CL316,243. However, despite that, the proportion of Wnt⁺ adipocytes in iWAT appeared unaltered in response to cold stress or β3-adrenergic receptor agonist treatments. Remarkably, as cold exposure was prolonged to 4 days, the number of Wnt⁻ beige adipocytes increased dramatically, but the majority of, if not all, UCP1-labeled Wnt⁻ beige adipocytes were found present neighboring Wnt⁺ adipocytes. UCP1⁺/Wnt⁻ beige adipocytes were rarely seen away from Wnt⁺ adipocytes within iWAT, implicating a regulatory role of Wnt⁺ adipocytes in beige fat biogenesis. Together with the results from mitochondrial activity assays, these observations suggest a paracrine function of Wnt⁺ adipocytes in modulating beige fat formation.

To determine whether those Wnt⁺ beige adipocytes are converted directly from Wnt⁺ adipocytes under cold conditions, lineage-tracing studies were conducted using Tcf/Lef-CreERT2;Rosa26RmTmG mice. After pre-treatment with tamoxifen and a 2-week washout at ambient temperature, Tcf/Lef-CreERT2;Rosa26RmTmG mice were subjected to cold challenge for 4 days. These mice manifested the presence of mGFP and UCP1 co-labeled adipocytes in iWAT, providing unambiguous evidence for the cell autonomous contribution of Wnt⁺ adipocytes to beiging.

To establish a non-cell autonomous role for Wnt⁺ adipocytes in beige fat biogenesis, a Fabp4-Flex-DTA mouse model was generated to provide a diphtheria-toxin-induced depletion system that allows targeted ablation of Wnt⁺ adipocytes upon crossing to Tcf/Lef-CreERT2 mice (“T/L-DTA mice”) and tamoxifen administration. Wnt⁺ adipocyte-ablated mice and littermate controls (Fabp4-Flex-DTA mice) were then challenged by 6° C. temperature. The core body temperature of each mouse was measured and it was found that about 31% T/L-DTA mice (5 of 16 mice) developed hypothermia (below 34.5° C.) within 60 hours, with the rest of the T/L-DTA mice exhibiting comparable core body temperature as the controls. The mice were further studied to determine if the formation of cold-induced beige fat in iWAT of T/L-DTA mice would be blunted. After 4-day cold stress, DTA-induced ablation of Wnt⁺ adipocytes led to substantially reduced UCP1-expressing beige adipocytes compared to control littermates. This markedly attenuated UCP1 expression persisted even after 2 weeks of cold adaptation. The compromised beiging process in iWAT was verified by remarkably decreased expression levels of thermogenic genes, as assessed by qRT-PCR and western blotting. Since the mRNA levels of the pan-adipogenic marker Adipoq (Adiponectin) appeared similarly between the genotypes, it was used as an internal reference. Loss of Wnt+ adipocytes in T/L-DTA mice caused significantly suppressed expression of thermogenesis-related genes specifically, but not those of general adipogenic markers in iWAT. Because Akita mice lack Wnt⁺ adipocytes in iWAT, similar to T/L-DTA mice, Akita mice were tested to determine whether they also display an impaired adaptive thermogenic capacity, by challenging Akita mice at 6° C. Immunofluorescence staining further validated that cold-induced UCP1+ cells were largely absent in iWAT of Akita mice. Collectively, these results demonstrate an essential role of Wnt⁺ adipocytes in initiating beige adipogenesis under cold conditions via non-cell autonomous effect.

### Wnt⁺ Adipocytes Enhance Systemic Glucose Handling in Mice

Given that the presence of Wnt⁺ adipocyte is highly dependent on insulin signaling, the potential physiological impact of Wnt⁺ adipocytes on whole-body glucose homeostasis was evaluated. “Loss-of-function” studies were conducted using T/L-DTA mice, which, under regular chow diet, were subjected to glucose tolerance assays 24 hrs after tamoxifen administration. The results showed clearly that T/L-DTA mice displayed impaired glucose handling competence as compared to controls despite similar basal glucose levels, indicating a requirement for Wnt⁺ adipocytes in handing systemic glucose. Next, a “gain-of-function” approach was used to test if Wnt⁺ adipocytes are sufficient to enhance systemic glucose utilization. For this purpose, immortalized GFPpos and mBaSVF cells were induced to become adipogenic commitment after 2-day in pro-adipogenic medium. Committed cells were mixed with Matrigel® and implanted into the left abdomen subcutaneous layer of wild-type C57BL/6J mice under regular chow. These implanted cells readily differentiated into fully mature adipocytes and formed vascularized adipose tissues in vivo within 2 weeks. Glucose tolerance assays were performed on mice 2 weeks after cell implantation. The results showed that mice bearing implanted Wnt⁺ adipocytes exhibited a significantly enhanced glucose tolerance as compared to mice receiving mBaSVF-derived adipocytes. Taken together, these results provide appealing evidence for the beneficial impact of Wnt⁺ adipocytes in systemic glucose homeostasis.

### Example 3

This Example discusses the results set forth in Example 2.

Over the past few decades, numerous studies support the notion that the canonical Wnt signaling pathway functions as a powerful suppressor of adipogenesis. The studies presented here challenge the conventional dogma by presenting direct evidence for the existence of a population of Wnt/β-catenin signaling driven adipocytes, namely Wnt⁺ adipocytes, revealing the diversity of adipocytes. The studies reported here further demonstrate that Wnt/β-catenin signaling is activated intracellularly via signaling crosstalk, in this case, AKT/mTOR signaling. Although if AKT/mTOR signaling cascade can activate canonical Wnt signaling remains contradictory, it appears to be cell-type dependent. This population of Wnt⁺ adipocytes was neglected, presumably because most of the previous studies were performed in aggregates of mixed cell types through Wnt ligand- and receptor-dependent manners. Importantly, the studies reported here show that intracellular Wnt/β-catenin signaling indeed has an overall impact on adipogenesis, as supported by its requirement for the survival of Wnt⁺ adipocytes and maturation of Wnt⁻ adipocytes. Since AKT/mTOR signaling is known to promote lipid storage and the studies reported here show it is required for the activation of the intracellular Wnt/β-catenin signaling, it is possible that Wnt⁻ adipocytes also possess basal activity Wnt/β-catenin signaling that is critical for lipid accumulation. This hypothesis is consistent with a prior study that silencing Tcf7l2 leads to blockade of adipogenesis.

The single-cell sequencing data developed in the course of the studies reported here showed that Wnt⁺ adipocytes, as compared to Wnt⁻ adipocytes induced from SVF cells of iWAT and iBAT origins, exhibit distinct signatures at the molecular and genomic levels, further supporting Wnt⁺ adipocytes as a unique subpopulation of fat cells in mammals. However, since FACS-based selection of mature adipocytes presents a challenge, sequencing was performed on in vitro cultured cells that were not under their true physiological status. Single nucleus RNA sequencing (snRNA-seq) was attempted on mature adipocytes isolated from adipose tissues. However, while others have reported successful studies in which adipocyte nuclei were isolated from adipose tissues simply by one-step enzyme digestion prior to snRNA-seq, the studies attempted here were stymied by nuclear membrane breakage after enzyme digestion and following FACS separation of Wnt⁺ and Wnt⁻ adipocyte nuclei. Nevertheless, under the standard white fat pro-adipogenic induction, SVF-induced Wnt⁺ adipocytes, but not Wnt⁻ adipocytes, express several brown/beige fat-selective genes, mirroring the thermogenic feature. In addition, pathway analyses indicate highly enriched insulin signaling pathway in Wnt⁺ adipocytes, as compared to Wnt⁻ fat cells, consistent with the intracellular activation of Wnt/β-catenin signaling by AKT/mTOR signaling cascade. While many subpopulations of heterogeneous pre-adipocytes/adipocytes have been identified, the classification of each subset replies mainly on the expression of specific genes such as cell surface markers (e.g., Oguri et al., Cell, 2020, 182(3):563-577.e20; Schwalie, et al., Nature, 2018, 559(7712):103-108). The studies reported here, based on the stable feature of an intrinsic cellular signaling identity, provide a new avenue for taxonomy of adipocytes.

In line with their thermogenic feature, as revealed by gene expression profiling, the studies reported herein have further uncovered a key role of Wnt⁺ adipocytes in adaptive thermogenesis of adipose tissues. First, Wnt⁺ adipocytes represent at least one of the cell populations that can convert into beige adipocytes directly following cold stress. Interestingly, a conversion of mature white adipocyte to reprogrammed beige characteristics has been postulated to occur predominantly in the “dormant” or “latent” beige fat (i.e., thermogenic inactive state of UCP1 lineage adipocytes within WAT depots), which was thought to form initially through de novo beige adipogenesis from progenitors during postnatal development. Because UCP1 expression within iWAT starts at postnatal day 14 independent of environmental temperature, dormant beige adipocytes appear much later in development than Wnt⁺ adipocytes, which can be found in the embryonic stage.

Therefore, together with the observations that Wnt⁺ and Wnt⁻ adipocytes do not convert mutually in cell culture, the findings reported herein suggest that Wnt⁺ adipocytes differ from dormant beige cells and represent a population of adipocytes that retain cellular plasticity and are capable of converting to beige adipocytes directly. Second, it is worth noting that Wnt⁺ adipocytes are also required for beige adipogenesis. Upon cold exposure, UCP1⁺/Wnt⁻ beige fat emerges largely surrounding Wnt⁺ adipocytes, implicating that Wnt⁺ adipocytes serve as a beiging initiator in a paracrine manner Remarkably, using a targeted cell ablation system, the studies herein showed that depletion of Wnt⁺ fat cells in mice leads to failed beige biogenesis in iWAT in response to cold stress. Severely impaired beiging phenotype was also seen in iWAT of Akita mice that lack Wnt⁺ adipocytes. These observations underscore the functional importance of Wnt⁺ adipocytes in triggering adaptive thermogenic program of adipose tissue. Despite that the critical factors produced by Wnt⁺ adipocytes to trigger beige adipogenesis remain unknown, the studies reported herein indicate that Wnt⁺ adipocytes act as a key initiator in beige biogenesis through both cell autonomous (direct conversion) and non-cell autonomous (recruitment) paths.

The significant contributions of beige adipocytes to improving whole-body glucose homeostasis and countering diet-induced obesity have been well documented (see, e.g., Bartelt and Heeren, Nat Rev Endocrinol. 2014; 10(1):24-36. doi: 10.1038/nrendo. 2013.204.2014; Harms and Seale, Nat Med. 2013; 19(10):1252-63.doi: 10.1038/nm.3361). Genome Wide Association Studies (“GWAS”) have also linked genetic variations in TCF7L2, a key component of canonical Wnt pathway, to type 2 diabetes in humans (Chen et al., 2018; Voight et al., Nat Genet, 2010, 42:579-589, doi:10.1038/ng.609). The studies here were driven in part by the speculation that Wnt⁺ adipocytes, driven by insulin/AKT signaling and functioning as beiging initiator in mice, represent a population of beneficial adipocytes and hold therapeutic potential for treating metabolic diseases. The results reported here demonstrate whole-body glucose intolerance in mice with targeted ablation of Wnt⁺ adipocytes but notably enhanced glucose utilization in mice receiving implantation of exogenously induced Wnt⁺ adipocytes, highlighting Wnt⁺ adipocytes as a therapeutically appealing candidate of cell source to restore systemic glucose homeostasis.

### Example 4

This Example sets forth the materials and methods used in isolating extracellular vesicles (EVs) and administering them to Akita mice.

Isolation of Extracellular Vesicles (EVs) from Adipocytes Induced from Immortalized Cells

GFPpos and GFPneg precursor cell lines were subjected to MesenCult™ Adipogenic Differentiation cocktail for 2 days to induce Wnt⁺ and Wnt⁻ adipocytes, respectively, followed by another 2-day culture in DMEM (catalog no. 3002101, Hyclone), supplemented with 10% exosome-depleted FBS (product A2720803, Thermo Fisher Scientific), 5 μg/ml insulin (product 10516, Sigma), and 1% penicillin-streptomycin. Culture media were collected and centrifuged at 2000 g for 30 min, after which the supernatants were transferred into Amicon® Ultra-15 Centrifugal Filter Units with Ultracel-100K (product no. UFC910024, Millipore) and centrifuged at 5000 g for another 30 min. Subsequently, EVs were isolated from filtered supernatants using Total Exosome Isolation Reagent (catalog no. 4478359, Thermo Fisher Scientific). Pellets were resuspended in 100 μl Dulbecco's phosphate buffered saline and the concentration of protein was determined using the BCA method. The ultrastructure of EVs was analyzed under a transmission electron microscope (model H7500, Hitachi High-Tech Corp., Japan). Representative markers, CD9 and GM130, were detected by western blot analysis. To determine the size of purified EVs, dynamic light scattering measurement was performed using the Zetasizer Nano ZS90 system (Malvern Pananalytical Ltd, Malvern, UK).

### In Vivo EVs Treatment

For EV treatment, 40 μg EVs isolated from induced Wnt⁺ and Wnt⁻ adipocytes were dissolved in 100 μl DPBS and were then injected into recipient Akita mice via tail vein, respectively. Blood glucose levels were measured on 0 d, 1 d and 7 d post EV administration, as described above. For in vivo distribution analysis of EV treatment, EVs were stained with fluorescent dye DiI (Thermofisher, V22888) prior to injection. Twenty-four hrs after injection, mice were sacrificed, and liver, iWAT, eWAT, and muscle tissues were collected for immunofluorescent imaging.

### Example 5

This Example describes the results of studies treating Akita mice with extracellular vesicles from Wnt⁺ adipocytes.

The results discussed in Example 2 showed that Wnt⁺ adipocytes exert a positive impact on glucose handling, and could be used as a therapeutic agent for metabolic disorders. As noted in Example 2, Ins2Akita mice (“Akita mice”) spontaneously develop insulin-dependent diabetes and have long been used as an animal model for type 1 diabetes mellitus (T1DM). Since Akita mice bear impaired insulin secretion, and since the results in Example 2 show that the survival, and the possible function of Wnt⁺ adipocytes, rely on insulin signaling, we suspected that grafted Wnt⁺ adipocytes might not develop and survive and therefore might not be useful as therapeutic agents in individuals with T1DM.

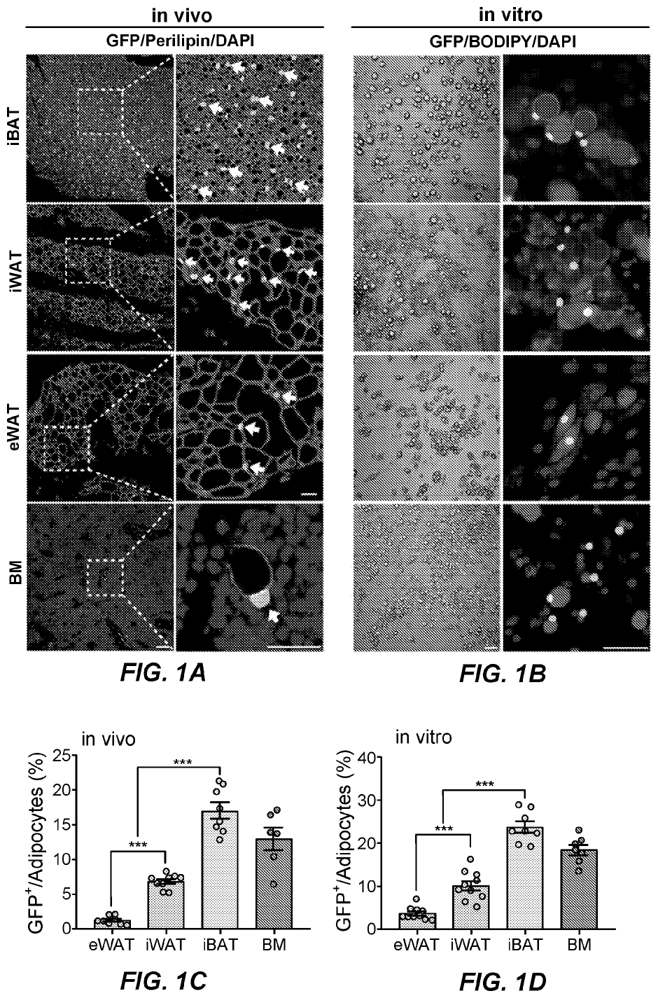

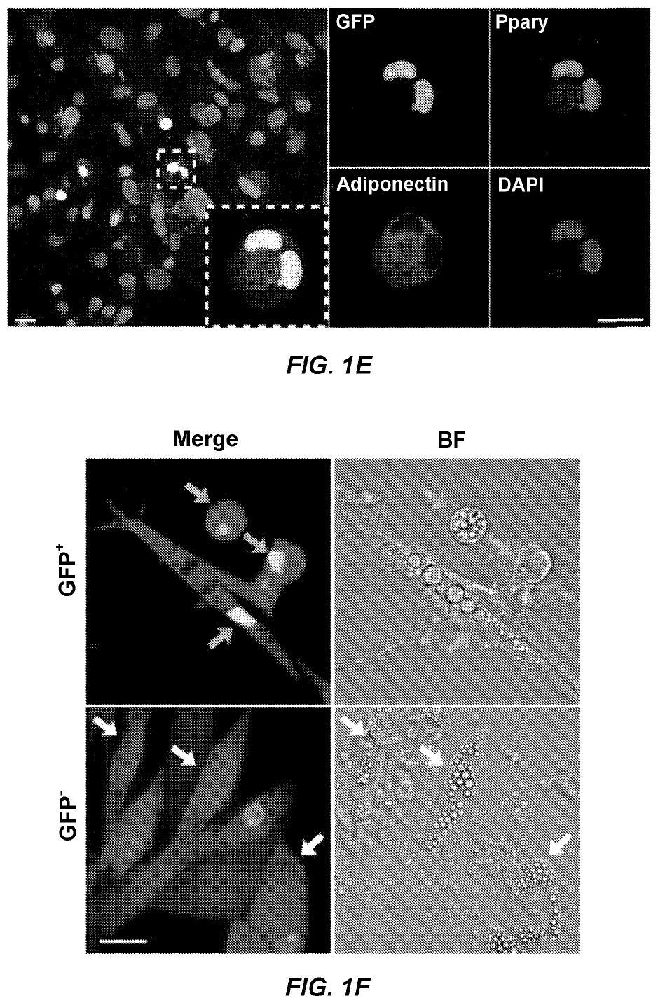

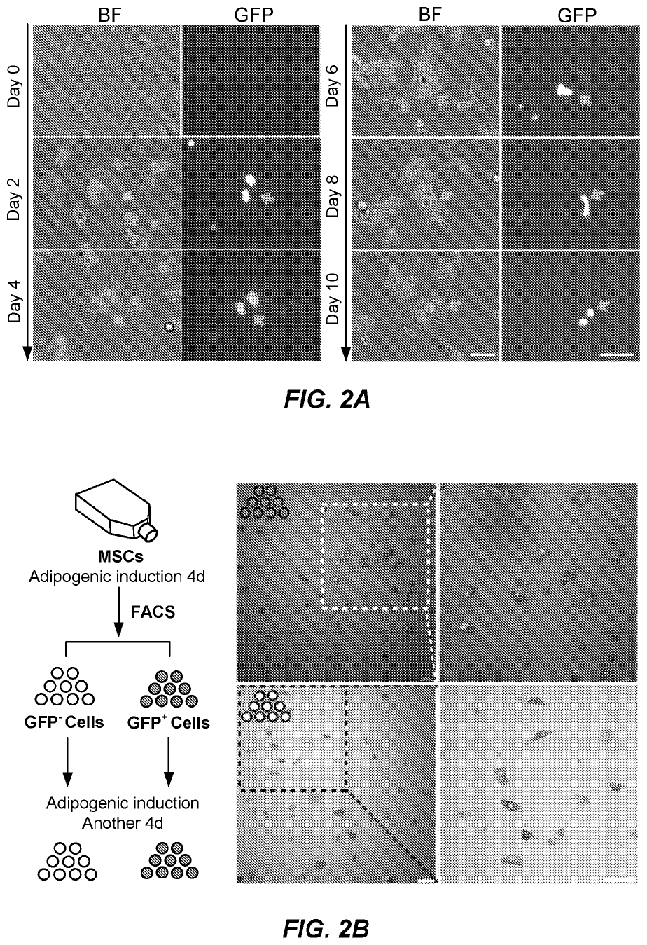

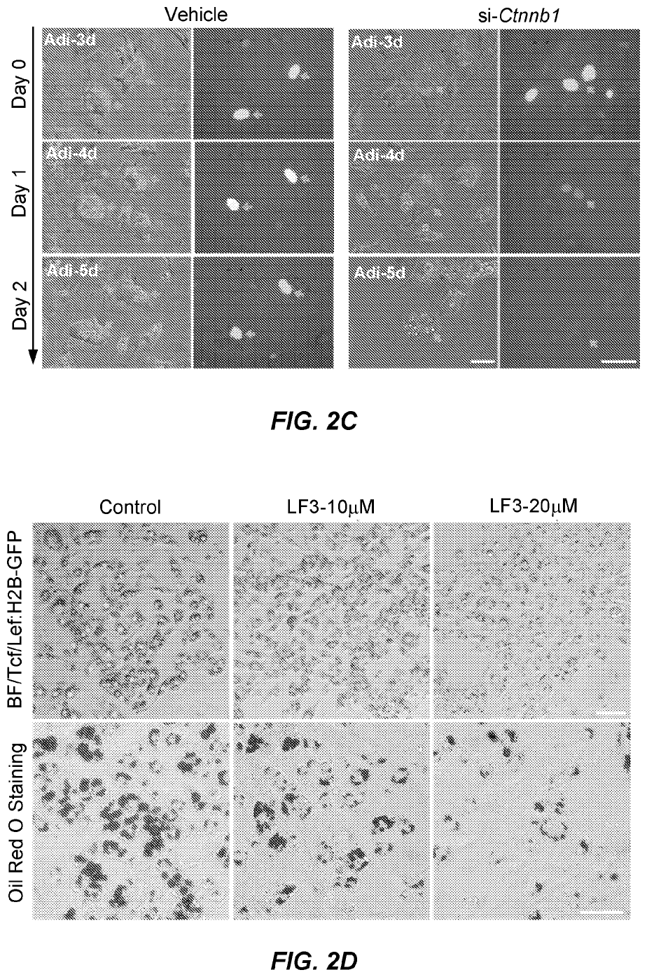

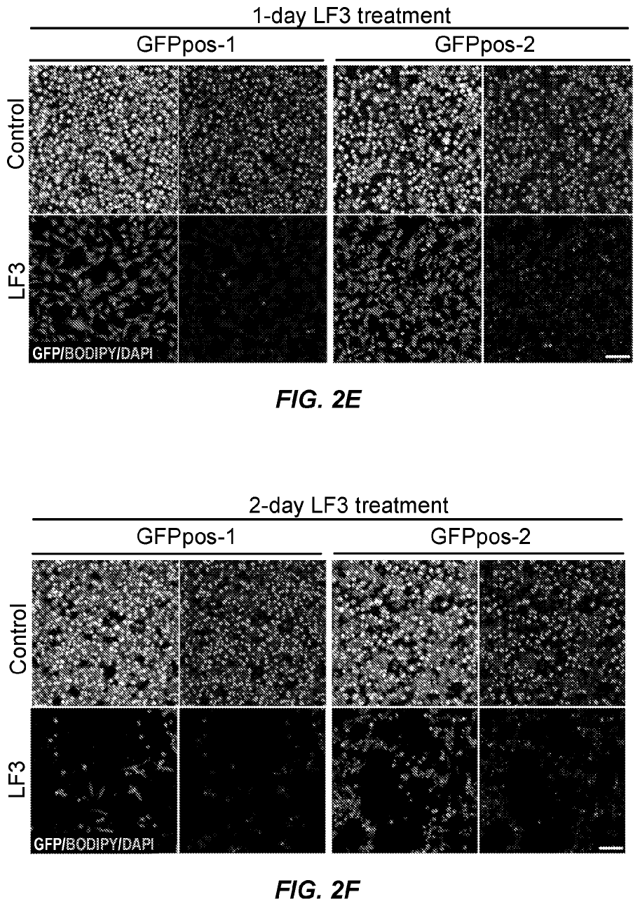

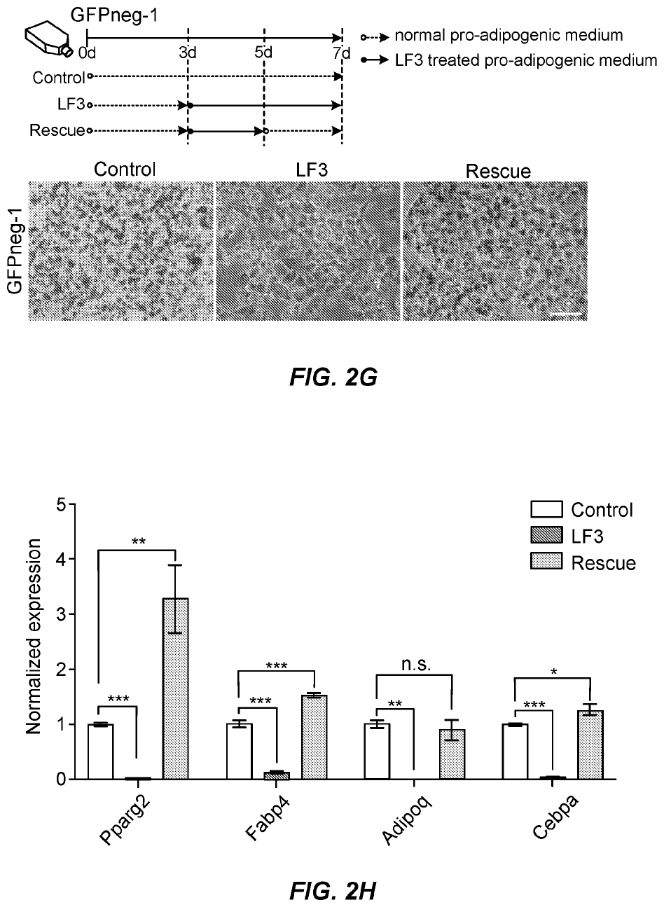

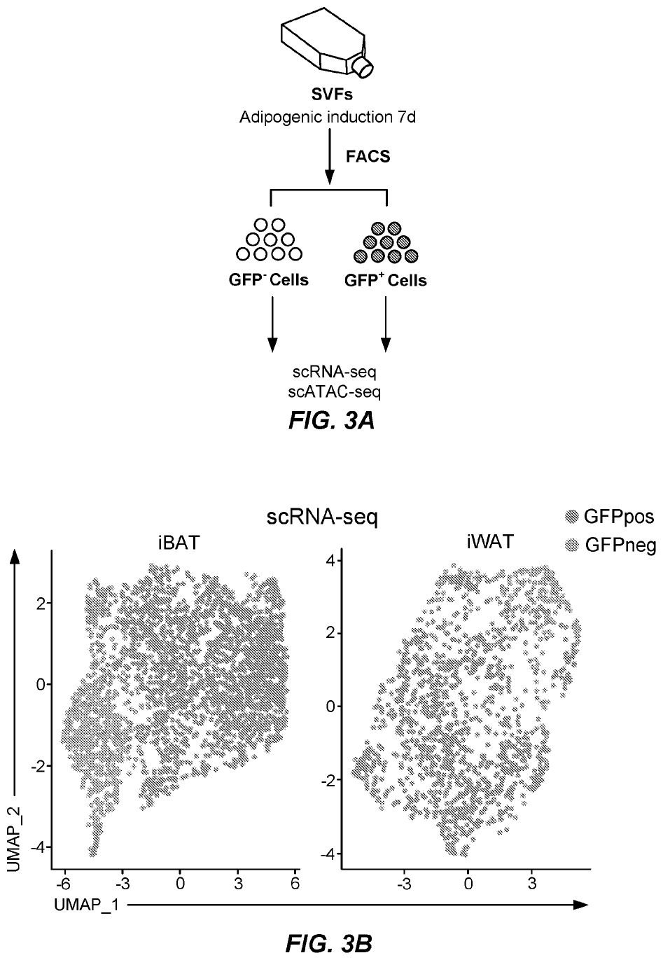

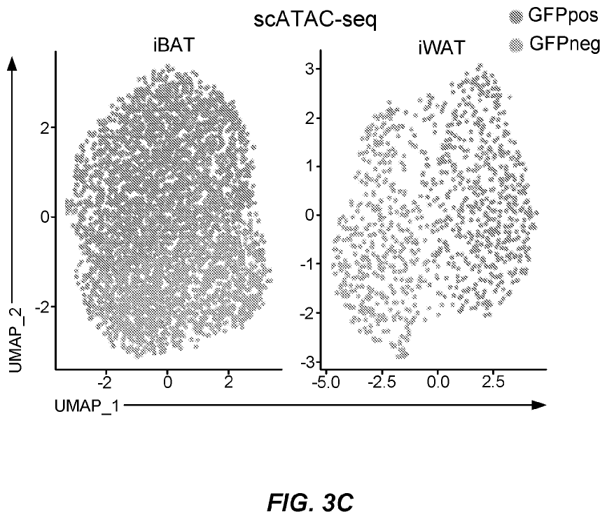

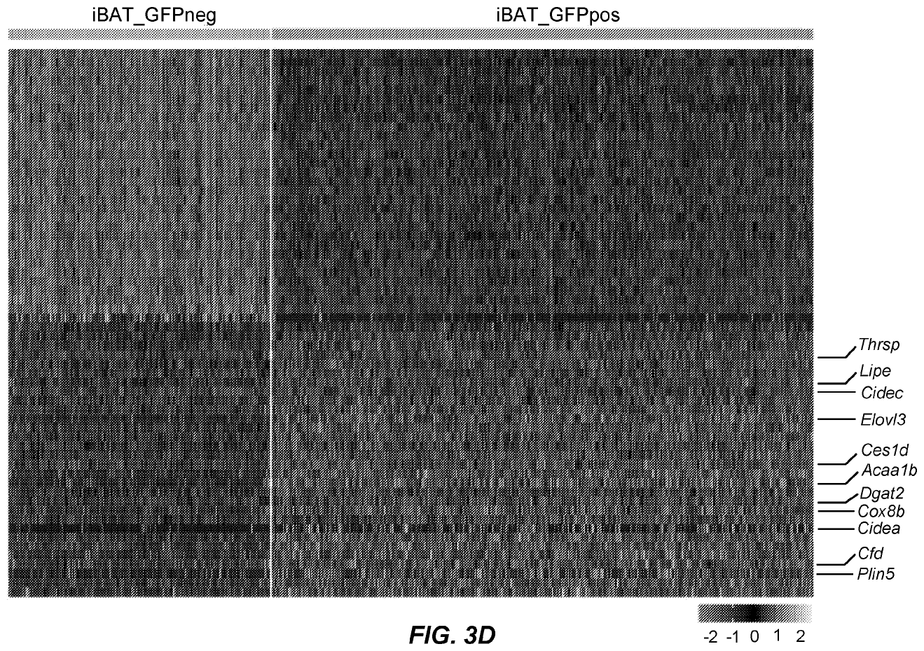

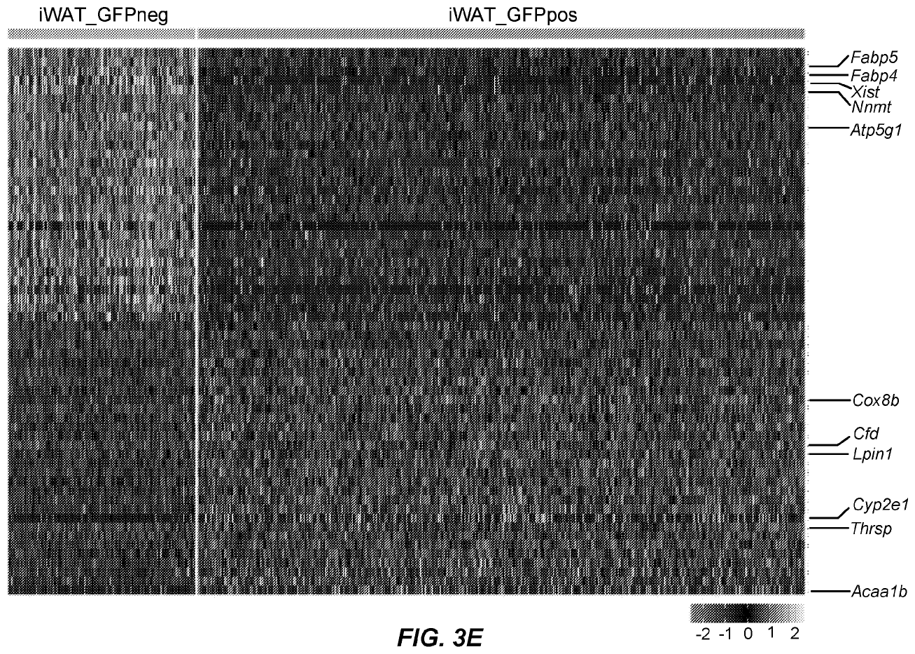

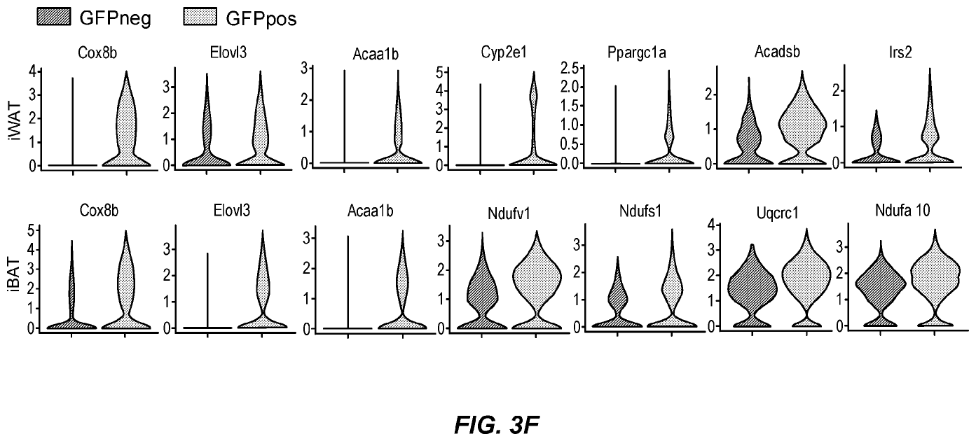

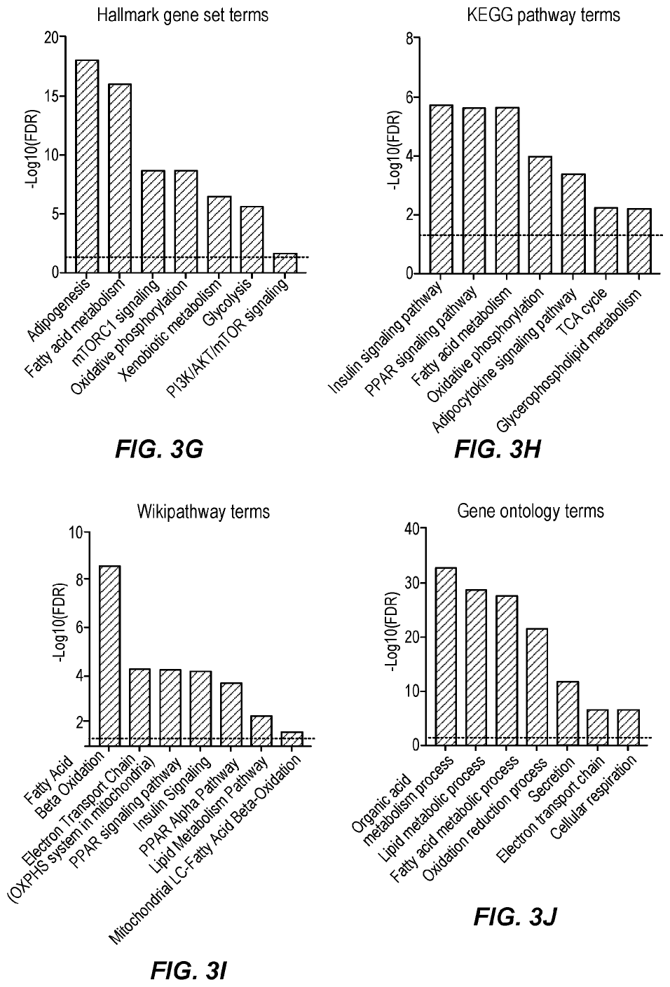

We decided to bypass this obstacle by using extracellular vesicles (EVs) from Wnt⁺ adipocytes, as multiple lines of evidence have pointed to the critical role of EVs in mediating circulating physiological function of adipose tissues in whole-body metabolic homeostasis. EVs were harvested from culture media of Wnt⁺ and Wnt⁻ adipocytes, respectively, induced from precursor cell lines and were injected into Akita mice through the tail vein (FIG. 1A-C). Strikingly, Akita mice treated with EVs from Wnt⁺ adipocytes but not those from Wnt⁻ adipocytes, manifested moderately but statistically significantly reduced basal glucose level within 24 hr after EV administration.

### Example 5

This Example describes the harvesting and isolation of human Wnt⁺ exosomes.

### A. Isolation of Human WAT SVFs.

Human subcutaneous white adipose tissue (WAT) samples can be obtained from healthy adult subjects during liposuction surgery under general anesthesia and frozen in liquid nitrogen and stored at −80° C. Stromal vascular fraction (SVF) cells from acquired WAT samples are isolated as described in the Examples above. In brief, fresh lipoaspirates are thawed on ice and washed twice with Dulbecco's phosphate buffered saline (DPBS) to remove visible connective tissue and blood. Fat tissues are dissected into ice-cold PBS and minced with scissors in a sterile 5 ml tube containing 4 ml of WAT digestion buffer (1×DPBS, 1% bovine serum albumin (BSA), 2.5 mg/ml dispase, and 4 mg/ml collagenase D) and incubated at 37° C. for 40 min under agitation. The digestion mixture is passed through a 100 μm cell strainer into a 50 ml tube. Digestion are stopped by adding 15 ml PBS containing 10% fetal bovine serum and centrifuged at 500 g for 5 min at room temperature. The supernatant is aspirated, and red blood cells are lysed by incubating the SVF pellet with 2 ml RBC lysis buffer (catalog no. 00433357, Invitrogen,™ Fisher Scientific Co. LLC, Pittsburgh, PA). The number and viability of cells in the suspension are determined using Trypan Blue stain according to the manufacturer's recommendations.

### B. Development of Immortalized Precursors of Human Wnt⁺ Adipocytes

To establish clonal cell lines of immortalized precursors of human Wnt⁺ and Wnt⁻ (as control) adipocytes, isolated primary human WAT SVFs are infected with lentivirus (LV) expressing SV40 Large T antigen. Lentiviral shuttle vector pUltra-hot-LT that expresses mCherry and Simian Virus 40 Large T antigen (SV40-LT) is generated simultaneously, as described in the preceding Examples. Plated human WAT SVF cells at passage 0 are transduced with pUltra-hot-LT LV particles. Two days after transduction, cells are purified by performing FACS to remove mCherry⁻ and Lin⁺ (CD31⁺CD45⁺TER119⁺) cells. The cells that are mCherry⁺ and Lin cells remaining are immortalized SVFs containing both Wnt⁺ and Wnt− adipocyte precursors.

To separate the immortalized precursors of human Wnt⁺ adipocytes, LV shuttle vector pUltra-hot-Tcf/Lef:H2B-GFP, which serves as Wnt/β-catenin signaling reporter, is used to infect immortalized SVFs as described in the preceding Examples, followed by hygromycin selection (150 mg/ml). After viral transduction, the cells are subjected to clonal selection through serial limited dilutions. The expression of green fluorescent protein (GFP) in cell colonies under pro-adipogenic induction is used as a readout for the accurate establishment of Wnt⁺ (>95%) adipocyte precursor cell lines. Wnt− adipocyte precursor cell lines will be isolated simultaneously as well.

C. Isolation of Exosomes/Extracellular Vesicles from Adipocytes Induced from Immortalized Human Wnt⁺ Cells

Immortalized human Wnt⁺ precursor cells are subjected to pro-adipogenic induction medium (catalog no. LL-0059, Lifeline Cell Technology) for 2 days to induce differentiation of Wnt⁺ adipocytes, followed by another 2-day culture in Dulbecco's Modified Eagle's Medium (DMEM) (product no. 3002101, Hyclone,™ Global Life Sciences Solutions USA LLC, Marlborough, MA), supplemented with 10% exosome-depleted FBS (catalog no. A2720803, Thermo Fisher Scientific, Waltham, MA), 5 μg/ml insulin (product no. 10516, Sigma Aldrich, Inc., St. Louis, MO), and 1% penicillin-streptomycin. Culture media are collected and centrifuged at 2000 g for 30 min, and then the supernatants are transferred into Amicon® Ultra-15 Centrifugal Filter Units with Ultracel-100K (product no. UFC910024, MilliporeSigma, Burlington, MA) and centrifuged at 5000 g for another 30 min. Subsequently, EVs are isolated from filtered supernatants using Invitrogen™ Total Exosome Isolation reagent (catalog no. 4478359, Thermo Fisher Scientific). Pellets will be resuspended in 100 μl DPBS and the concentration of protein be determined using the BCA method. The ultrastructure of EVs will be analyzed under a transmission electron microscope (model H7500, Hitachi High-Tech Corp., Japan). Representative markers, CD9 and GM130, will be detected by western blot analysis. To determine the size of purified EVs, dynamic light scattering measurement will be performed using the Zetasizer Nano ZS90 system (Malvern Pananalytical Ltd, Malvern, UK). For clinical trials, exosomes/EVs will be isolated from Wnt− precursor cell lines as controls using the same approach.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A population of exosomes derived from a population of adipocytes, at least 30% of which adipocytes are Wnt⁺ adiopocytes.

2. The population of exosomes of claim 1, wherein at least 50% of said population of adipocytes are Wnt⁺ adiopocytes.

3. The population of exosomes of claim 1, wherein at least 90% of said adipocytes are Wnt⁺ adiopocytes.

4. The population of exosomes of claim 1, wherein said population of Wnt⁺ adiopocytes are from white adipocytes.

5. The population of exosomes of claim 1, wherein said population of adipocytes are of human adipocytes.

6-11. (canceled)

12. A method of ameliorating symptoms of lack of insulin production or of insulin resistance in a subject in need thereof, said method comprising administering to said subject a composition comprising exosomes secreted by a population of adipocytes, at least 50% of which are Wnt⁺ adiopocytes.

13. The method of claim 12, wherein at least 75% of said population of adipocytes are Wnt⁺ adiopocytes.

14. The method of claim 12, wherein 90% or more of said population of adipocytes are Wnt⁺ adiopocytes.

15. The method of claim 12, wherein said subject in need thereof has type 1 diabetes mellitus.

16. The method of claim 12, wherein said subject in need thereof has type 2 diabetes mellitus.

17. The method of claim 12, wherein said exosomes are in a pharmaceutically acceptable carrier.

18. A composition comprising exosomes derived from a population of adipocytes, of which at least 50% of said population of adipocytes are Wnt⁺ adiopocytes.

19. The composition of claim 18, wherein at least 75% of said population of adipocytes are Wnt⁺ adiopocytes.

20. The composition of claim 18, wherein at least 90% of said population of adipocytes are Wnt⁺ adiopocytes.

21. The composition of claim 18, further comprising a pharmaceutically acceptable carrier, an excipient, or both a pharmaceutically acceptable carrier and an excipient.

22. (canceled)

23. A method for generating from human stem cells or stromal cells a population of adipocytes in which Wnt⁺ adiopocytes are present in a desired higher percentage than in naturally-occurring populations of adipocytes, said method comprising:
(a) introducing a Wnt/β-catenin reporter genetic construct into human stem cells or stromal cells of interest, which reporter genetic construct provides a detectable signal when Wnt/β-catenin signaling is present in said human stem cells or stromal cells, or in cells differentiated into adipocytes from said human stem cells or stromal cells,
(b) creating duplicate first and second cultures of said human stem cells or stromal cells of interest carrying said Wnt/β-catenin reporter genetic construct,
(c) subjecting said first culture of said human stem cells or stromal cells of interest carrying said Wnt/β-catenin reporter genetic construct to adipogenic induction,
(d) examining differentiated adipocytes in said first culture of said human stem cells or stromal cells subjected to said adipogenic induction for the presence of said detectable signal from said Wnt/β-catenin reporter genetic construct,
(e) expanding cells of said second culture of human stem cells or stromal cells if a detectable signal is detected in said first culture, and,
(f) repeating steps (c)-(e) until a population of adipocytes results which contains said population of adipocytes of which said desired higher percentage of Wnt⁺ adipocytes is present than in naturally-occurring populations of adipocytes,
thereby generating a population of adipocytes in which Wnt⁺ adiopocytes are present in a desired higher percentage than in naturally-occurring populations of adipocytes.

24. The method of claim 23, wherein said human stem cells or stromal cells of interest are bone marrow-derived stromal cells, adipose-derived stem cells, or umbilical cord stem cells.

25. The method of claim 23, wherein said human stem cells or stromal cells of interest are bone marrow-derived stromal cells.

26. The method of claim 23, in which said Wnt/β-catenin reporter genetic construct is Tcf.Lef:H2B-GFP or in which said sequence encoding GFP has been replaced by a sequence encoding red fluorescent protein or yellow fluorescent protein.

27. The method of claim 23, in which said desired higher percentage in which of Wnt⁺ adiopocytes are present than in naturally-occurring populations of adipocytes is 50%.

28. The method of claim 23, in which said desired higher percentage in which of Wnt⁺ adiopocytes are present than in naturally-occurring populations of adipocytes is 75%.

29. The method of claim 23, in which said desired higher percentage in which of Wnt⁺ adiopocytes are present than in naturally-occurring populations of adipocytes is 90%.

30. The method of claim 23, in which said desired higher percentage in which of Wnt⁺ adiopocytes are present than in naturally-occurring populations of adipocytes is 95% or higher.

31. The method of claim 23, in which said Wnt/β-catenin reporter genetic construct is introduced into said human stem cells or said stromal cells by transfection of said cells with a viral vector.

32. The method of claim 31, in which said transfection of said cells with a viral vector is by a lentiviral vector.

33. The method of claim 31, in which said transfection of said cells with a viral vector is by an adeno-associated viral vector.

34. The method of claim 23, in which said Wnt/β-catenin reporter genetic construct is introduced into said human stem cells or said stromal cells by CRISPR.

35. The method of claim 34, in which CRISPR is used to introduce said Wnt/β-catenin reporter genetic construct into said human stem cells or said stromal cells at a site referred to as Rosa26.

36. A method of ameliorating symptoms of insulin production or of insulin resistance in a subject in need thereof, said method comprising administering to said subject a composition comprising a population of adipocytes, at least 50% of which population of adipocytes are Wnt⁺ adiopocytes.

37. The method of claim 36, wherein at least 75% of said population of adipocytes are Wnt⁺ adiopocytes.

38. The method of claim 36, wherein at least 95% of said population of adipocytes are Wnt⁺ adiopocytes.

39. The method of claim 36, wherein said subject in need thereof has Type 1 diabetes mellitus, and said population of adipocytes is co-administered with insulin.

40. The method of claim 36, wherein said subject in need thereof has Type 2 diabetes mellitus.

41. The method of claim 36, wherein said population of adipocytes is in a pharmaceutically acceptable carrier.


SEQUENCE LISTING (4 sequences)

SEQ ID NO: 1 - DNA, 46 bp, Artificial sequence
  Primer
gactcatctagagataaagttttaaacagagaggaatctttgcagc

SEQ ID NO: 2 - DNA, 33 bp, Artificial sequence
  Primer 2
gcatacggatcctgtttcaggttcagggggagg

SEQ ID NO: 3 - DNA, 42 bp, Artificial sequence
  Primer 3
agagatccagtttggttaattaatattaaccctcactaaagg

SEQ ID NO: 4 - DNA, 38 bp, Artificial sequence
  Primer 4
tggagccgacacgggttaatttacttgtacagctcgtc